(19) United States
(12) Patent Application Publication
(10) Pub. No.: US 2006/0160210 A1
(43) Pub. Date: Jul. 20, 2006

(54) Biological sample analysis plate

(75) Inventors: Kazuyoshi Mori, Matsuyama-shi (JP); Motohiro Yamashita, Matsuyama-shi (JP); Toshifumi Nanjo, Touon-shi (JP)

Correspondence Address: STEPTOE & JOHNSON LLP, 1330 CONNECTICUT AVE., NW, WASHINGTON, DC, 20036, US

(73) Assignee: Matsushita Electric Industrial Co., Ltd., Kadoma-shi (JP)

(21) Appl. No.: 11/287,315

(22) Filed: Nov. 28, 2005

(30) Foreign Application Priority Data
Nov. 26, 2004 (JP) 2004-342215
Feb. 10, 2005 (JP) 2005-034029
Mar. 1, 2005 (JP) 2005-055545
Apr. 28, 2005 (JP) 2005-132007

(51) Int. Cl. C12M 1/34

(52) U.S. Cl. 435/287.2

(57) ABSTRACT

In a biological sample analysis plate, one ends of channels 11 and 12 are connected to a quantitative sorting part 10 having a predetermined volume for taking a predetermined amount of a biological sample. On the other end of the channel 11, a buffer agent storage part 7 for holding a buffer agent to be filled in the channels 11 and 12 is provided in a position that is concentric with the quantitative sorting part 10 with respect to an axial center of the plate. Further, an overflow chamber 17 into which excess buffer agent that cannot be stored in the buffer agent storage part flows is connected to the buffer agent storage part 7. Therefore, it is possible to provide a biological sample analysis plate which can facilitate injection of the buffer agent and the biological sample, and obtain accurate result of detection in a short time, when performing detection by making the biological sample migrate in the buffer agent filled in the channels.

### FIELD OF INVENTION

The present invention relates to a biological sample analysis plate in which a biological sample such as DNA, protein, or the like migrates in a buffer agent, and a transport reaction during the migration is detected to analyze the biological sample.

### BACKGROUND OF THE INVENTION

When considering general biological samples, DNA and protein exist broadly. In recent years, with rapid progress of chemical biology, involvement of genome in various diseases has been understood with a fair degree of precision, and medical care targeted at genome has attracted attention.

With respect to DNA, SNPs (Single Nucleotide Polymorphism which is a general term for a difference of a single code (a single nucleotide) in genome) attract attention presently. The reason is as follows. By classifying SNPs, it is possible to predict the prevalence rates of many diseases, and the effects or sensitivities of individuals to medical agents, and furthermore, it is possible to perform perfect identification of an individual because there never exist plural human beings having completely the same SNPs on the planet, even parent and child or brothers.

As a method for examining SNPs, “sequencing” (determination of base sequence) in which a DNA base sequence is directly read from an end, has been employed most commonly. As a method for performing sequencing, although several methods have been reported, “dideoxy sequencing” (Sanger method) has been carried out most commonly. Sequencing is, in any method including the Sanger method, established on the basis of a technique for separating/discriminating a difference in single-base lengths by modified polyacrylamide gel electrophoresis having a high separative power, or capillary electrophoresis.

As another method, there is affinity ligand capillary electrophoresis. The affinity ligand capillary electrophoresis gives specificity to separation, utilizing intermolecular affinity, especially, specific affinity in ecosystem (enzyme-substrate affinity or antigen-antibody affinity). To be specific, analysis is carried out while focusing attention on a phenomenon that, when a sample migrates by electrophoresis in an electrophoresis solution stored in a capillary tube, to which an affinity ligand that specifically recognizes a base sequence is added, only molecular species that mutually react in the sample mixture have variations in migration speed (for example, refer to Japanese Published Patent Application No. Hei. 7-311198 (Patent Document 1)).

On the other hand, proteins exist in cells, tissues, and bio-fluids, and are involved in control of organic activities, supply of energies to cells, combination of important substances, maintenance of organic structures, and further, inter-cell communication and intra-cell information transmission. Recently, it becomes increasingly clear that proteins have plural functions according to various environments, existence of other proteins for mutual reaction, degrees and kinds of modifications given to proteins.

A protein is produced by connecting 20 kinds of amino acids in the order according to a genetic instruction (sequence information), and it is said that there are tens of millions of proteins. If the genetic sequence is found, it is possible to obtain information as to what amino acids are connected in what order. A set of proteins produced by biotic genomes is called “proteome”, and analysis of proteome has been increasingly carried out with completion of sequencing of human genome.

In analyzing and studying the functions of proteins, it is necessary to perform, not only identification and characterization, but also biochemical assay, investigation for inter-protein reactions, elucidation of protein network, and elucidation of signaling in and out of cells. Various fields of technologies are employed in studying the protein functions, for example, enzyme assay, yeast two-hybrid assay, purification by chromatography, information tool and data base, and the like. Particularly, discrimination of proteins by electrophoresis is an important scheme. There are various reports relating to fluid transportation and orientation in the case where sample analysis, discrimination, determination, and the like are carried out by detecting a transport reaction obtained when migrating a fluid such as a sample, an analyte, a buffer agent, or a reagent in a capillary tube such as electrophoresis (for example, refer to Japanese Published Patent Application No. 2000-513813 (Patent Document 2), and Japanese Published Patent Application No. 2001-523341 (Patent Document 3)).

As described above, the method utilizing a capillary electrophoresis apparatus has been widely used for biological sample analysis. A glass tube having an outer diameter of about 300μ and an inner diameter of about 100μ is often used as a capillary which is a part of actually performing a transport reaction, and its surface is coated with polyimide or the like to make it tough.

However, a portion of the coating should be removed by burning or melting with a chemical to produce a detection window for detecting a sample inside the tube. At this time, the portion where the coating is removed becomes frangible, and therefore, careful handling is required. If the tube is broken, it is dangerous.

Further, injection of a sample is generally carried out by pressurization or aspiration. Since the sample must be injected by a predetermined amount, the injection is controlled by time. However, the injection amount varies from experiment to experiment because of variations in the viscosity and temperature of the buffer agent in the capillary. Since the amount of the sample significantly affects the measurement result, it is very important item.

Furthermore, in the apparatus using such capillary, it is difficult to carry out electrophoresis in a short channel because of the structure of the apparatus, and therefore, measurement should be carried out with excessive electrophoresis distance and time.

In order to simplify the complicated work mentioned above, there is a method of electrophoresis using a plate in which minute channels are formed, instead of the capillary.

However, even when utilizing the channels on the plate, sample separation should be carried out as follows. For example, in the methods described in Patent Document 2 and 3, plural capillary channels in which a buffer agent for trapping a sample is filled are intersected, and at least three electrodes are provided. A voltage is applied to two electrodes among the at least three electrodes, thereby to make the sample migrate through the intersection point. In this method, however, since the channels intersect, there is a possibility that the sample cannot migrate well when it performs electrophoresis. Furthermore, since separated samples are not constant but vary with variations in the viscosity and temperature of the buffer agent, accurate measurement result cannot be obtained.

### SUMMARY OF THE INVENTION

The present invention is made to solve the above-mentioned problems and has for its object to provide a biological sample analysis plate which requires no complicated preparation, and provide accurate detection result in short time, when performing detection of a biological sample by making the sample migrate in a buffer agent that is filled in channels.

According to the present invention, there is provided a biological sample analysis plate having a channel in which an injected buffer agent is filled by a centrifugal force that is generated by rotating the plate about its axial center, the channel comprising a first channel and a second channel, one ends of the first and second channels being connected to a quantitative sorting part having a predetermined volume for taking a predetermined amount of a biological sample, and the first and second channels having a shape extending from the quantitative sorting part toward an outer circumference with respect to the axial center; and the biological sample analysis plate includes a buffer agent storage part for holding the buffer agent that is filled in the channel, the buffer agent storage part being located at the other end of the first channel, in a position on a concentric circle with the quantitative sorting part with respect to the axial center, or in a position closer to the axial center than the quantitative sorting part.

According to the present invention, the second channel is connected to an overflow chamber into which excess buffer agent that cannot be stored in the buffer agent storage part flows.

According to the present invention, an upper-level buffer agent injection part for injecting the buffer agent is provided in a position closer to the axial center than the buffer agent storage part, and the upper-level buffer agent injection part and the buffer agent storage part are connected to each other.

According to the present invention, a through-hole in which air comes in and out is provided in the channel or in the upper-level buffer agent injection part, thereby to promote filling of the buffer agent into the channel.

According to the present invention, a biological sample injection part for injecting the biological sample is connected to the quantitative sorting part, and a through-hole in which air comes in and out is provided in the quantitative sorting part so as to promote supply of the biological sample held by the biological sample injection part into the quantitative sorting part.

According to the present invention, the quantitative sorting part is connected to an overflow chamber into which the biological sample that is supplied over a predetermined amount flows.

According to the present invention, there is provided a biological sample analysis plate in which a quantitative sorting part for introducing a predetermined amount of a biological sample into a buffer agent channel is provided at a junction of a sample channel in which the biological sample flows and the buffer agent channel in which a buffer agent flows, the plate performing analysis of the biological sample by detecting a transport reaction that occurs when the biological sample introduced in the quantitative sorting part migrates in the buffer agent; wherein the sample channel is provided on an inner circumference side of a closed channel which forms the buffer agent channel.

According to the present invention, the length of a portion of the buffer agent channel, which portion is formed from the center of the biological sample analysis plate toward the outer circumference, is longer than the length of a portion of the buffer agent channel, which portion is formed in a rotation direction of the plate around the center of the plate.

According to the present invention, the buffer agent channel has a rectangle shape.

According to the present invention, the buffer agent channel is disposed on a concentric circle with the gravity center of the biological sample analysis plate as a center of rotation.

According to the present invention, the cross-sectional area of a portion of the buffer agent channel on the inner circumference side is larger than the cross-sectional area of a portion of the buffer agent channel on the outer circumference side.

According to the present invention, there is provided a biological sample analysis plate for detecting a transport reaction that occurs when a biological sample migrates in a buffer agent, thereby to analyze the biological sample, and the biological sample analysis plate comprises a quantitative sorting part having a predetermined volume for holding the biological sample, which is provided at a junction of a third channel in which the buffer agent flows and a fourth channel in which the biological sample flows, and a suppression means for suppressing flow of the biological sample held by the quantitative sorting part into the fourth channel, which is provided in the quantitative sorting part.

According to the present invention, a cross-sectional area of a portion of the fourth channel for making the biological sample flow into the quantitative sorting part is equal to or larger than a cross-sectional area of a portion of the fourth channel for making the biological sample flow from the quantitative sorting part.

According to the present invention, a cross-sectional area of a portion of the fourth channel which is disposed above the suppression means in the quantitative sorting part is equal to or smaller than a cross-sectional area of a portion between the suppressing means and a side wall surface of the quantitative sorting part, which portion makes the biological sample flow from the fourth channel into the quantitative sorting part, and is equal to or smaller than a cross-sectional area of a portion between the suppressing means and the side wall surface of the quantitative sorting part, which portion makes the biological sample flow from the quantitative sorting part to the fourth channel.

According to the present invention, the quantitative sorting part is provided with a guide means for guiding the biological sample into the third channel.

According to the present invention, the guide means is a protruding portion of the quantitative sorting part.

According to the present invention, the cross-sectional area of the third channel decreases in proportion to the distance from the quantitative sorting part.

According to the present invention, there is provided a biological sample analysis plate for detecting a transport reaction that occurs when a biological sample migrates in a buffer agent, thereby to perform analysis of the biological sample, and the biological sample analysis plate comprises a fifth channel in which the biological sample flows, a sixth channel in which the buffer agent flows, and a quantitative sorting part for holding a predetermined volume of the biological sample, which part is disposed at a junction of the fifth channel and the sixth channel; wherein, among channels at the junction of the quantitative sorting part and the sixth channel, a cross-sectional area of a sample flow-in channel through which the biological sample flows from the quantitative sorting part to the sixth channel decreases in proportion to the distance from the quantitative sorting part.

According to the present invention, the quantitative sorting part comprises plural parts, and the biological sample flows from the plural parts into the flow-in channel.

According to the present invention, the sixth channel comprises plural channels.

Therefore, according to the present invention, a predetermined amount of the biological sample can be reliably added to the buffer agent in a short time by a centrifugal force, whereby accurate detection can be carried out in the subsequent electrophoresis. Further, when supplying the buffer agent and the biological sample into the plate, it is possible to supply just enough amounts of the buffer agent and the biological sample which are required for analysis, by only injecting the buffer agent and the sample so as to approximately fill the entire buffer agent injection part and biological sample injection part, respectively.

Consequently, when the buffer agent and the biological sample are supplied from a pipette or the like to the plate, it is possible to inject the buffer agent and the sample by simple operation, without requiring practiced operation for discharging predetermined amounts of the buffer agent and the biological sample. Thereby, it is possible to provide a biological sample analysis plate which does not need complicated preparation, and can obtain accurate detection result in a short time.

### BRIEF DESCRIPTION OF THE DRAWINGS

FIG. 1 is a plan view of a biological sample analysis plate 100 according to a first embodiment of the present invention.

FIG. 2 is an enlarged plan view of a pattern of the biological sample analysis plate 100.

FIG. 3 is a cross-sectional view of a sample injection part and a buffer agent injection part of the biological sample analysis plate 100.

FIG. 4 is a plan view illustrating a pattern of the biological sample analysis plate 100.

FIG. 5 is an enlarged plan view of a quantitative sorting part of the biological sample analysis plate 100.

FIG. 6(a) is a plan view illustrating a pattern of the biological sample analysis plate 100.

FIG. 6(b) is a plan view illustrating a pattern of the biological sample analysis plate 100.

FIG. 7 is a plan view illustrating a pattern of the biological sample analysis plate 100.

FIG. 8 is an enlarged plan view of the quantitative sorting part of the biological sample analysis plate 100.

FIG. 9 is a plan view illustrating a pattern of a biological sample analysis plate 200 according to a second embodiment of the present invention,

FIG. 10 is a diagram illustrating a groove formation surface of a biological sample analysis plate 300 according to a third embodiment of the present invention.

FIG. 11(a) is a diagram illustrating a pattern formed on the biological sample analysis plate 300 according to the third embodiment.

FIG. 11(b) is a diagram illustrating a surface on which grooves are formed in a channel pattern in which sample channels and buffer agent channels are concentrically arranged, according to the third embodiment.

FIG. 12 is a cross-sectional view of a sample injection part and a buffer agent injection part of the biological sample analysis plate 300 according to the third embodiment.

FIG. 13 is a cross-sectional view of a chamber part, a sample holding part, and a buffer part of the biological sample analysis plate 300 according to the third embodiment.

FIG. 14 is a cross-sectional view of a positive electrode and a negative electrode of the biological sample analysis plate 300 according to the third embodiment.

FIG. 15(a) is a diagram illustrating the state of the DNA conjugate when the biological sample analysis plate 300 of the third embodiment is subjected to a sample filling process.

FIG. 15(b) is an enlarged view of a quantitative sorting part illustrating the state of the DNA conjugate when the biological sample analysis plate 300 according to the third embodiment is subjected to the sample filling process.

FIG. 16 is a diagram illustrating the states of the respective samples when the DNA conjugate and the DNA sample are filled in the biological sample analysis plate 300 of the third embodiment and the plate 300 is rotated at 4000 rpm.

FIG. 17 is a diagram illustrating the states of the respective samples when the DNA conjugate and the DNA sample are filled in the biological sample analysis plate 300 of the third embodiment, and the plate 300 is rotated at 4000 rpm, and thereafter, the rotation is suddenly stopped.

FIG. 18 is a diagram illustrating the states of the respective samples when the DNA conjugate and the DNA sample are filled in the biological sample analysis plate 300 of the third embodiment, and the plate 300 is rotated at 4000 rpm and then stopped, and thereafter, rotation is again started.

FIG. 19 is a diagram illustrating a part for performing DNA scanning on the biological sample analysis plate 300 of the third embodiment.

FIG. 20 is a diagram illustrating how the DNA sample, which is applied to the quantitative sorting part formed on the biological sample analysis plate 300 of the third embodiment, migrates in the DNA conjugate for separation which is filled in the first channel.

FIG. 21 is a diagram illustrating the shape of a quantitative holding part and its vicinity on a biological sample analysis plate 400 according to a fourth embodiment of the present invention.

FIG. 22 is a diagram for explaining the construction of the biological sample analysis plate 400 according to the fourth embodiment.

FIG. 23 is a diagram for explaining the construction of the biological sample analysis plate 400 according to the fourth embodiment.

FIG. 24(a) is a diagram illustrating an upper surface of the plate 400 according to the fourth embodiment.

FIG. 24(b) is a diagram illustrating a lower surface of the plate 400 according to the fourth embodiment.

FIG. 24(c) is a diagram illustrating a D-D cross-section of the plate 400 according to the fourth embodiment.

FIG. 25 is a block diagram illustrating a biological sample determination apparatus 1000 according to the fourth embodiment.

FIG. 26 is a flowchart illustrating a sequence of operations of the biological sample determination apparatus 1000 according to the fourth embodiment.

FIG. 27(a) is a diagram illustrating a pattern formed on the plate 400 of the fourth embodiment, at a point in time when a sample is injected into the pattern.

FIG. 27(b) is a diagram illustrating the pattern formed on the plate 400 of the fourth embodiment, at a point in time when the pattern is subjected to a conjugate filling process.

FIG. 27(c) is a diagram illustrating the pattern formed on the plate 400 of the fourth embodiment, after the pattern is subjected to the conjugate filling process.

FIG. 27(d) is a diagram illustrating the pattern formed on the plate 400 of the fourth embodiment, after the pattern is subjected to pressurization.

FIG. 27(e) is a diagram illustrating the pattern formed on the plate 400 of the fourth embodiment, after the pattern is subjected to a quantitative adding process.

FIG. 28(a) is a diagram illustrating an elevation stage at its first-level position, in the biological sample determination apparatus 1000 according to the fourth embodiment.

FIG. 28(b) is a diagram illustrating the elevation stage which is moving from the first-level stage to the second-level stage, in the biological sample determination apparatus 1000 according to the fourth embodiment.

FIG. 28(c) is a diagram illustrating the elevation stage which is moving from the first-level stage to the second-level stage, in the biological sample determination apparatus 1000 according to the fourth embodiment.

FIG. 28(d) is a diagram illustrating the elevation stage at the second-level position, in the biological sample determination apparatus 1000 according to the fourth embodiment.

FIG. 29 is a diagram illustrating a signal from a plate fitting position detection sensor of the biological sample determination apparatus 1000 according to the fourth embodiment.

FIG. 30(a) is a diagram illustrating an example of positional relationship between a thermistor and a heater of the biological sample determination apparatus 1000 according to the fourth embodiment.

FIG. 30(b) is a diagram illustrating another example of positional relationship between the thermistor and the heater of the biological sample determination apparatus 1000 according to the fourth embodiment.

FIG. 31 is a diagram illustrating how the DNA sample, which is applied to the second channel formed on the biological sample analysis plate 400 of the fourth embodiment, migrates in the DNA conjugate for separation which is filled in the first channel.

FIG. 32 is a diagram illustrating a measurement result of absorbance of the DNA sample, in the biological sample determination apparatus 1000 according to the fourth embodiment.

FIG. 33 is a diagram for explaining the principle of the biological sample determination apparatus 1000 according to the fourth embodiment.

FIG. 34 is a diagram illustrating a lower surface of the biological sample analysis plate 400 of the fourth embodiment, on which four patterns are disposed.

FIG. 35 is a diagram illustrating a pattern formation surface of a biological sample analysis plate 500 according to a fifth embodiment of the present invention.

FIG. 36 is a diagram illustrating a sample injection surface of the biological sample analysis plate 500 according to the fifth embodiment.

FIG. 37 is a diagram illustrating a pattern formed on the biological sample analysis plate 500 according to the fifth embodiment.

FIG. 38 is a diagram illustrating the states of the respective samples when the DNA conjugate and the DNA sample are filled in the biological sample analysis plate 500 of the fifth embodiment and the plate 500 is rotated at 4000 rpm.

FIG. 39 is a diagram illustrating the states of the respective samples when the DNA conjugate and the DNA sample are filled in the biological sample analysis plate 500 of the fifth embodiment, and the plate 500 is rotated at 4000 rpm, and thereafter, the rotation is suddenly stopped.

FIG. 40 is a diagram illustrating the states of the respective samples when the DNA conjugate and the DNA sample are filled in the biological sample analysis plate 500 of the fifth embodiment, and the plate 500 is rotated at 4000 rpm and then stopped, and thereafter, rotation is again started.

FIG. 41 is a diagram illustrating how the DNA sample, which is applied to a sample quantitation part formed on the biological sample analysis plate 500 of the fifth embodiment, migrates in the DNA conjugate for separation which is filled in the first channel.

FIG. 42 is a diagram viewed from a channel formation surface of a biological sample analysis plate 600 according to a sixth embodiment of the present invention.

FIG. 43 is an enlarged view of a sample quantitation part and its vicinity of the biological sample analysis plate 600 according to the sixth embodiment.

FIG. 44 is an enlarged view of a sample quantitation part and its vicinity, illustrating the DNA sample migration state after starting electrophoresis, in the biological sample analysis plate 600 according to the sixth embodiment.

FIG. 45 is an enlarged view of the sample quantitation part and its vicinity, illustrating the DNA sample migration state after starting electrophoresis, in the biological sample analysis plate 600 according to the sixth embodiment.

### DETAILED DESCRIPTION OF THE PREFERRED EMBODIMENTS

### Embodiment 1

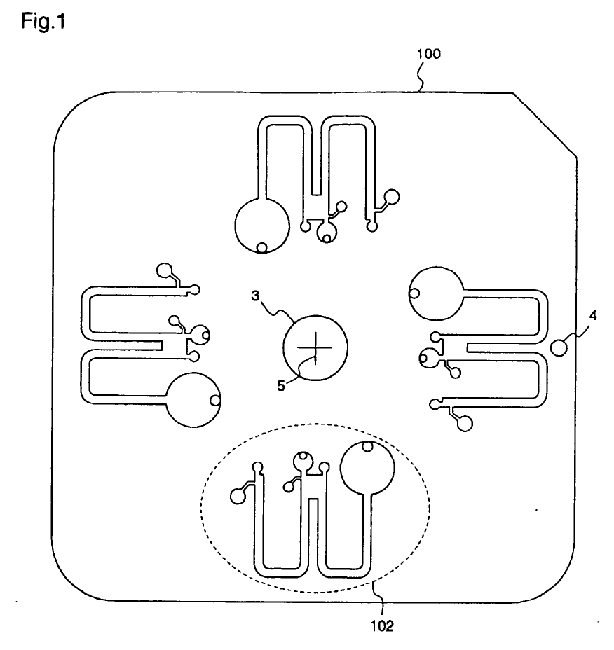

Hereinafter, a biological sample analysis plate according to a first embodiment of the present invention will be described with reference to FIGS. 1˜8.

In the present invention, a biological sample is transferred in a buffer agent to carry out biological, enzymatical, immunological, and chemical reactions, thereby to analyze the biological sample easily, inexpensively, accurately, and speedily.

In this first embodiment, in order to specify the description, it is assumed that the biological sample is a DNA sample and the buffer agent includes a DNA conjugate for separation and a DNA bonding control agent. The biological sample analysis plate adds a predetermined quantity of the DNA sample into the DNA conjugate for separation which is filled in a channel to make the sample perform electrophoresis in the conjugate, and detects fluorescence or absorbance in the channel to determine presence/absence of SNPs (Single Nucleotide Polymorphism) in the DNA sample.

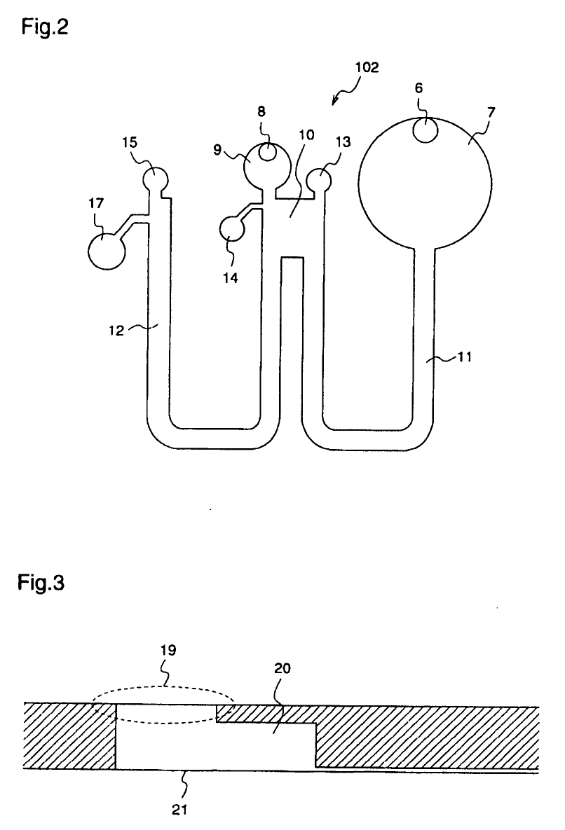

Initially, the construction of the biological sample analysis plate according to the first embodiment will be described with reference to FIGS. 1˜3. FIG. 1 is a diagram illustrating the biological sample analysis plate viewed from a channel formation surface, according to the first embodiment. FIG. 2 is a diagram illustrating a specific shape of a pattern 2 which is formed on the plate 1 shown in FIG. 1.

On the biological sample analysis plate 100 according to the first embodiment, four patterns identical to the pattern 2 shown in FIG. 2 are radially fabricated, so that DNA analysis for four analytes can be carried out simultaneously. As shown in FIG. 1, the outer shape of the plate 100 according to the first embodiment is an 8-cm square, the four corners thereof are chamfered, and one of the four corners is especially largely chamfered. A hole 4 is formed to make the outer shape of the plate asymmetrical so that the positions of the patterns can be specified.

The reason why the plate 100 has such outer shape is because the outer shape can facilitate positioning when detecting fluorescence or absorbance with the plate being fixed to an optical reading device (not shown). The plate 100 comprises an acrylic plastic, and it is 2 mm thick. Further, grooves are formed on the channel formation surface, and an acrylic film having a thickness of 50 μm is adhered onto the surface, thereby producing hermetically closed channels.

Reference numeral 5 denotes an axial center of the plate 100, and numeral 3 denotes a hole for fixing the plate 100 to a rotation member of the optical reading device with the axial center 5 in the center. In FIG. 2, reference numeral 6 denotes a buffer agent inlet also serving as an air hole, through which DNA conjugate as a buffer agent is injected, numeral 7 denotes a buffer agent injection part for temporarily holding the injected buffer agent, numeral 8 denotes a sample inlet also serving and an air hole, through which a DNA sample is injected, and numeral 9 denotes a sample injection part for temporarily holding the injected DNA sample. The buffer agent injection part 7 and the sample injection part 8 have similar shapes, and a cross-sectional view of the part 7 or 8 and its vicinity is shown in FIG. 3.

FIG. 3 is a diagram illustrating a cross-section of the buffer agent injection part 7 or the sample injection part 9 and its vicinity, wherein diagonally hatched portions correspond to the plate 100. In FIG. 3, reference numeral 19 denotes the buffer agent inlet 6 or the sample inlet 8, and numeral 20 denotes the buffer agent injection part 7 or the sample injection part 9. Reference numeral 21 denotes the above-mentioned film, and a closed channel is formed by adhering the film so as to cover the grooves. In FIG. 3, the channel formation surface is on the lower side.

Turning to FIG. 2, the sample injection part 9 is directly connected to a sample quantitative sorting part 10, and the buffer agent injection part 7 is also connected to the sample quantitative sorting part 10 through the channel 11. Further, the quantitative sorting part 10 is connected to an air hole 13 for air releasing, an overflow chamber 14 for quantifying the injected DNA sample by a centrifugal force, and a channel 12 for performing scanning by the above-mentioned optical reading device during electrophoresis. At an end of the channel 12 for optical scanning, an air hole 15 is provided, and further, an overflow chamber 17 is disposed, into which excess buffer agent that cannot be stored in the buffer agent storage part flows. In this first embodiment, the channels 11 and 12 are 50 μm wide and 50 μm deep.

Next, a description will be given of examples of specific operation and action until presence/absence of SNPs (Single Nucleotide Polymorphism) in the DNA sample is determined, with reference to FIG. 2 showing the specific shape of the outer circumference side pattern 2.

Initially, a DNA sample as an analyte is prepared. Essentially, DNA has a duplex-strand helical structure. In this first embodiment, however, there is prepared single-strand DNA having a base length of about 60 bases including SNPs to be discriminated. Since extraction and denaturation methods are not directly related to the present invention, specific description thereof will be omitted.

Next, a DNA conjugate is prepared as a buffer agent. A DNA conjugate is obtained by covalently bonding a high-molecular linear polymer to a 5′ end of single-strand DNA having a base length of 6˜12 bases. Further, the DNA conjugate has a sequence that is complemental to normal DNA but non-complemental to mutant DNA, and the bonding force of the DNA conjugate to the normal DNA is strong while the bonding force of the DNA conjugate to the mutant DNA is weak. Further, when performing electrophoresis, the electrophoresis speed is considerably low because the linear polymer bonded to the 5′ end acts as a weight. It is assumed that “DNA conjugate” described hereinafter is a material containing a pH buffer agent that also serves as an electrolyte, and a DNA bonding force control agent such as MgCl₂.

When preparation of the samples is completed, the DNA conjugate and the DNA sample are injected into the plate 100. Initially, the DNA conjugate is dispensed from the buffer agent inlet 6 using a pipeter or the like so as to fill the buffer agent injection part 7. Although the amount of the DNA conjugate to be dispensed depends on the scale of pattern, 3 microliters of the DNA conjugate is required to fill the buffer agent injection part 7, the buffer agent channel 11, the quantitative sorting part 10, and the channel 12 for optical scanning. The buffer agent injection part 1 is previously formed so as to have a volume a little larger than 3 microliters.

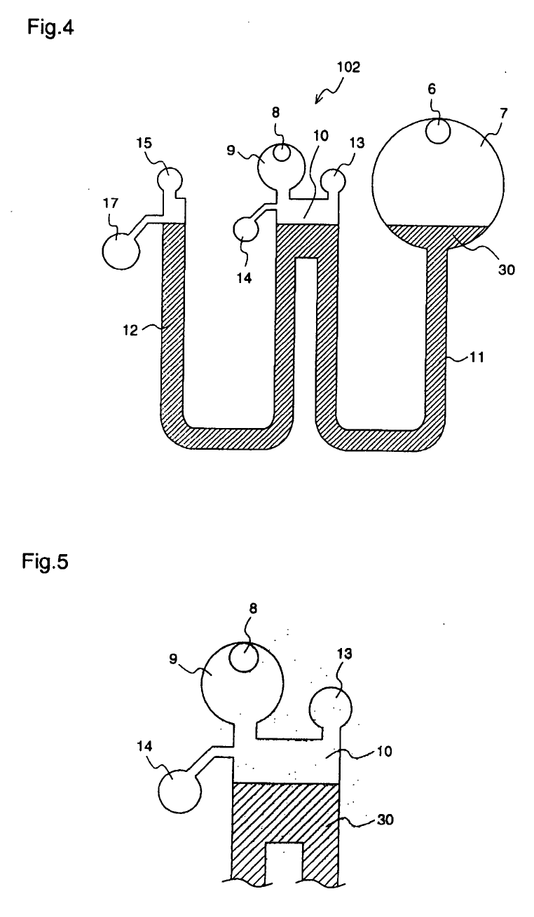

Next, the plate 100 is fixed to the rotation part of the optical reading apparatus, and is rotated around the axial center 5. At this time, the dispensed DNA conjugate migrates toward the outer circumference due to a centrifugal force. The DNA conjugate in the buffer agent injection part 7 migrates through the channel 11 to the quantitative sorting unit 10, and thereafter, reaches the channel 12. FIG. 4 shows the state where migration of the DNA conjugate is stopped two minutes after starting rotation at 4000 rpm. Further, FIG. 5 is an enlarged view of the quantitative sorting part 10 and its vicinity.

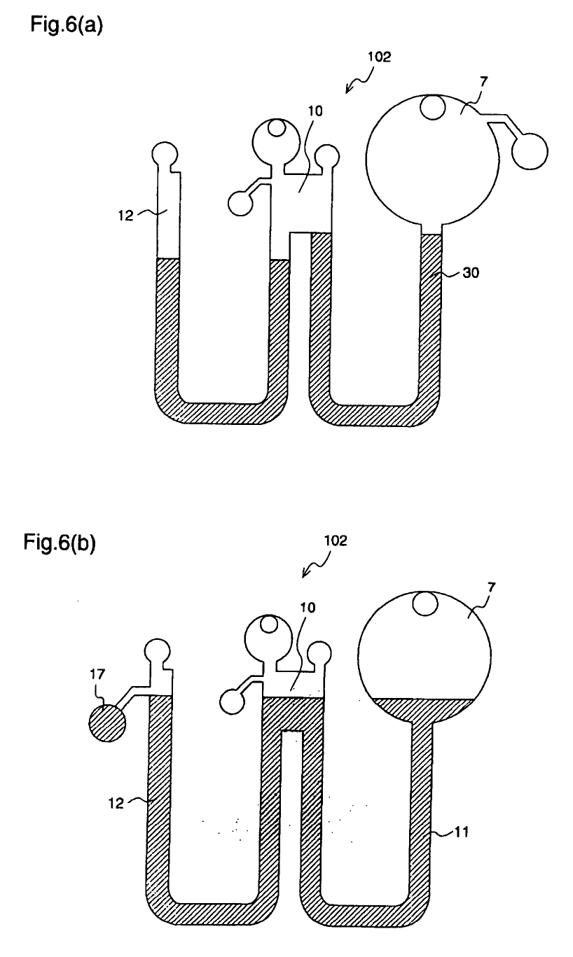

When migration of the DNA conjugate has ended, the fluid level of the DNA conjugate 30 existing in the buffer agent injection part 7 and the optical scanning channel 12 and the fluid level of the DNA conjugate 30 existing in the quantitative sorting part 10 are on the same circle with the axis 5 in the center. However, when the quantity of the DNA conjugate injected into the buffer agent injection part 7 is smaller than 3 microliters, no DNA conjugate is filled in the quantitative sorting part 10 and the channel 12 as shown in FIG. 6(a). No electrophoresis is carried out when no DNA conjugate is filled in the quantitative sorting part 10 and the channel 12.

On the other hand, for the case where the quantity of the DNA conjugate 30 injected into the buffer agent injection part 7 is larger than 3 microliters, the overflow chamber 17 into which excess buffer agent that cannot be stored in the buffer agent storage part flows is connected to the second channel 12. Therefore, the fluid level of the DNA conjugate 30 existing in the buffer agent injection part 7 and the optical scanning channel 12 and the fluid level of the DNA conjugate 30 existing in the quantitative sorting part 10 are always on the same circle with the axis 5 in the center. As shown in FIG. 6(b), the overflow chamber 17 is disposed in a position on the channel 12, on the same circle with the axis 5 in the center when the volume of the hatched portions excluding the overflow chamber 17, i.e., the volume of the hatched portions of the buffer agent injection part 7, the buffer channel 11, the quantitative sorting part 10, and the optical reading scanning channel 12 becomes 3 microliters.

Next, after the DNA conjugate is supplied into the channel, the DNA sample is injected into the plate 100. The DNA sample is dispensed from the sample inlet 8 into the sample injection part 9 using a pipeter or the like. Since, in this first embodiment, 1 microliter of DNA sample is needed, the sample injection part 9 is formed so as to have a volume a little larger than 1 microleter.

Next, the plate 100 is fixed to the rotation part, and rotated around the axis 5. At this time, the dispensed DNA sample migrates toward the outer circumference due to a centrifugal force.

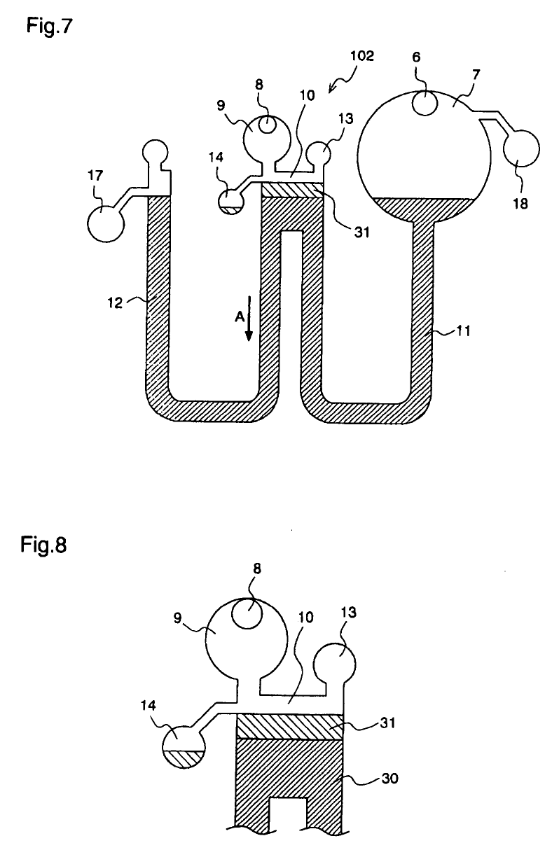

The DNA sample in the sample injection part 9 migrates to the quantitative sorting part 10 without occurrence of bubbles due to the air hole 13. At this time, the DNA sample in this quantitative sorting part 10 can be quantified by the overflow chamber 14, and unnecessary DNA sample migrates into the overflow chamber 14. FIG. 7 shows the state where migration of the DNA sample is stopped two minutes after starting rotation at 4000 rpm. In FIG. 7, reference numeral 18 denotes an overflow chamber connected to the buffer agent injection part 7. FIG. 8 is an enlarged view of the quantitative sorting part 10 and its vicinity.

In FIGS. 7 and 8, reference numeral 31 denotes the DNA sample remaining in the quantitative sorting part 10.

Through the above-mentioned operation, the DNA sample 31 remaining in the quantitative sorting part 10 becomes the final sample to be subjected to discrimination of SNPs.

Next, electrophoresis is carried out. The film 21 is adhered to the entire surface of a portion of the front surface of the plate where the channels are disposed, and a needle-shaped electrode is thrust onto the film 21 and inserted into the plate, thereby to enable power application. A positive electrode is inserted in an end of the channel 12 for optical scanning, while a negative electrode is inserted in the buffer agent injection part 7. As for electrophoresis, when a voltage of several hundreds volts is applied between the electrodes, electric fields occur in the optical scanning channel 12 as well as in the quantitative sorting part 10, whereby a predetermined amount of the DNA sample that remains in the quantitative sorting part 10 migrates toward the positive electrode side (A direction in FIG. 7) in the optical scanning channel 12.

The optical scanning channel 12 is filled with the DNA conjugate, and the DNA sample performs electrophoresis while repeating bonding with the DNA conjugate. At this time, since bonding force of the normal DNA in the DNA sample with the DNA conjugate in strong, the electrophoresis speed is low, while the bonding force of the mutant DNA with the DNA conjugate is weak, the electrophoresis speed is high. That is, when both the normal DNA and the mutant DNA exist in the DNA sample, the normal DNA and the mutant DNA are separated from each other, whereby discrimination of SNPs can be carried out.

As described above, the biological sample analysis plate 100 according to the first embodiment is provided with the channel in which the injected buffer agent is filled by a centrifugal force that is generated by rotating the plate 100 about its axial center, and the channel is composed of the first channel 11 and the second channel 12, one ends of which are connected to the quantitative sorting part 10 having a predetermined volume for taking a predetermined amount of the biological sample, and each of the first and second channels has a shape extending toward the outer circumference with respect to the axial center from the quantitative sorting part 10. Further, at the other end of the first channel 11, the buffer agent storage part for holding the buffer agent to be filled in the channel is provided in a position on a concentric circle with the quantitative sorting part 10 with respect to the axial center, or in a position closer to the axial center than the quantitative sorting part 10. Therefore, when supplying the buffer agent to the plate, just enough amounts of the buffer agent required for analysis can be supplied to the channel as well as the buffer agent can be injected so as to fill the entire buffer agent injection part. Accordingly, in the biological sample analysis plate for analyzing the biological sample by making the biological sample remaining in the quantitative sorting part perform electrophoresis, when supplying the buffer agent to the plate from a pipette or the like, it is possible to inject the buffer agent by a simple operation without requiring a practiced operation for discharging only a predetermined amount of the agent, thereby providing a biological sample analysis plate that can be easily handled.

Furthermore, the second channel 12 is provided with the overflow chamber 14 into which excess buffer agent that cannot be stored in the buffer agent storage part flows, and only a predetermined amount of the biological sample 31 is added to the buffer agent stored in the quantitative sorting part 10. Therefore, the biological sample can reliably perform electrophoresis in the buffer agent, whereby accurate analysis can be carried out in a short time without requiring complicated preparation.

### Embodiment 2

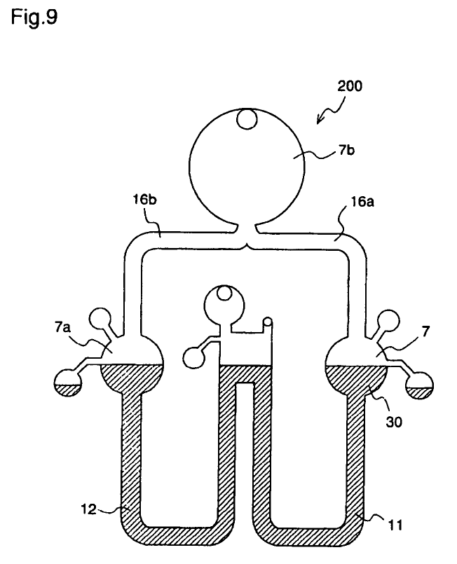

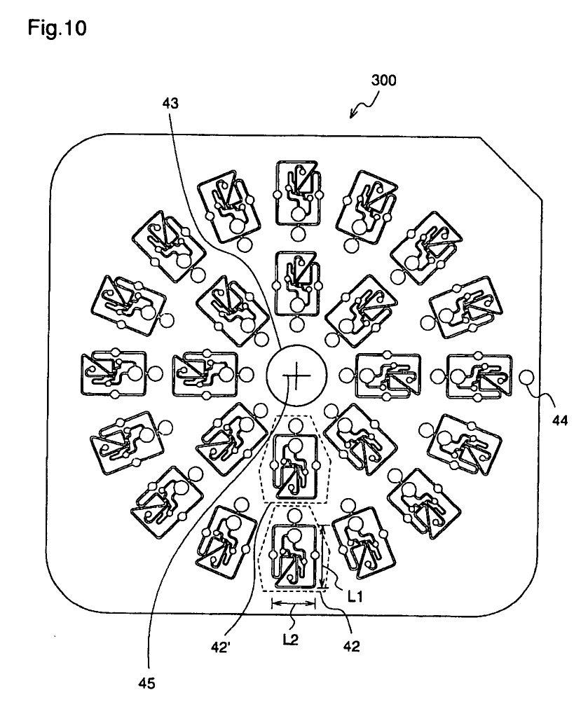

Next, a biological sample analysis plate according to a second embodiment of the present invention will be described with reference to FIG. 9.

The biological sample analysis plate according to the second embodiment is different from the biological sample analysis plate according to the first embodiment in that not an air hole 15 but a buffer agent injection part 7a is provided at an end of the optical scanning channel 12, and further, an upper-level buffer agent injection part 7b for supplying a buffer agent to both of the buffer agent injection parts 7 and 7a is connected to the buffer agent injection part 7 and the buffer agent injection part 7a through a channel 16. Hereinafter, only the difference from the first embodiment will be described.

With reference to FIG. 9, the biological sample analysis plate 200 according to the second embodiment is provided with the upper-level buffer agent injection part 7b which is connected to the buffer agent injection part 7 and the buffer agent injection part 7a through the channels 16a and 16b, respectively. Therefore, when injecting the DNA conjugate 30 into the plate 200, it is only required to inject the DNA conjugate 30 into the upper-level buffer agent injection part 7b only. Further, when the plate 200 is rotated at 4000 rpm, the migration time of the DNA conjugate due to a centrifugal force is shorter than 2 minutes, and the migration can be completed in 1 minute and 40 seconds. Furthermore, in the construction of the biological sample analysis plate 200 according to the second embodiment, filling of the buffer agent is carried out from the upper end portions of the left and right channels 11 and 12, simultaneously. Therefore, filling of the DNA conjugate can be carried out reliably without forming air bubbles or the like by making the volumes of the channel 11 and the channel 12 approximately equal to each other.

As described above, according to the biological sample analysis plate of the second embodiment, the buffer agent injection part 7a is provided at the end portion of the optical scanning channel 12, and the upper-level buffer agent injection part 7b for supplying the buffer agent to both of the buffer agent injection parts 7 and 7a are connected to the buffer agent injection part 7 and the buffer agent injection part 7a through the channels 16a and 16b, respectively. Therefore, injection and filling of the DNA conjugate into the plate 200 can be done by only injecting the buffer agent into the upper-level buffer agent injection part 7b alone, and further, the injection can be reliably carried out in a short time without forming air bubbles or the like.

### Embodiment 3

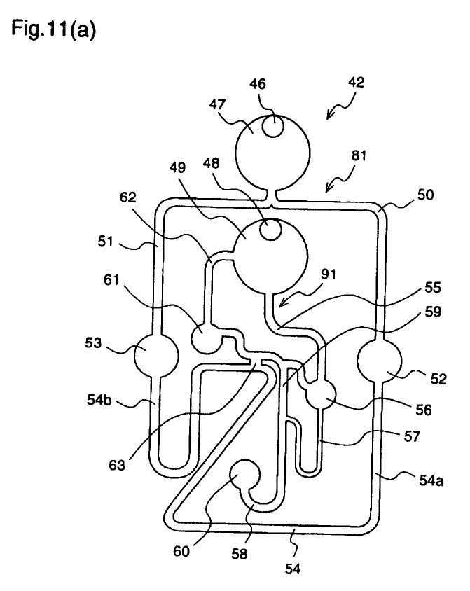

Next, a biological sample analysis plate according to a third embodiment of the present invention will be described with reference to FIGS. 10˜20.

According to the present invention, a biological sample is migrated in a buffer agent to carry out biological, enzymatical, immunological, and chemical reactions, thereby to analyze the biological sample easily, inexpensively, accurately, and speedily.

In this third embodiment, in order to specify the description, it is assumed that the biological sample is a DNA sample, and the buffer agent includes a DNA conjugate for separation and a DNA bonding control agent. The biological sample analysis plate adds a predetermined quantity of the DNA sample into the DNA conjugate for separation which is filled in a channel to make the sample perform electrophoresis, and detects fluorescence or absorbance in the channel, thereby to determine presence/absence of SNPs (Single Nucleotide Polymorphism) in the DNA sample.

Initially, the construction of the biological sample analysis plate 300 according to the third embodiment will be described with reference to FIGS. 10˜14. FIG. 10 is a diagram illustrating the biological sample analysis plate viewed from its channel formation surface, according to the third embodiment.

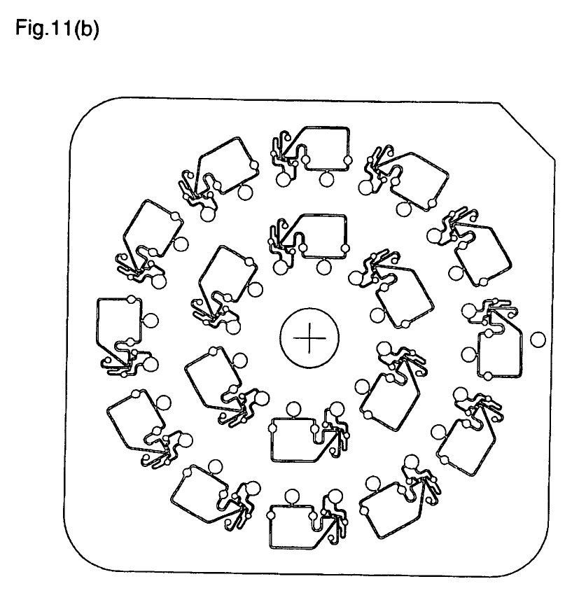

FIG. 11a is a diagram illustrating a detailed shape of an outer circumference side pattern 2 formed on the biological sample analysis plate 300 shown in FIG. 10.

On the biological sample analysis plate 300 according to the third embodiment, 16 outer circumference side patterns 42, which are identical in shape to the pattern shown in FIG. 11a, are radially formed, and 8 inner circumference side patterns 42′, which are identical in shape to the outer circumference side patterns 42, are radially formed at the inner side of the outer circumference side patterns 42. The outer circumference side patterns 42 and the inner circumference side patterns 42′ enable DNA analysis for 24 analytes at the same time. The outer circumference side patterns 42 and the inner circumference side patterns 42′ are disposed concentrically around the gravity center of the biological sample analysis plate 41.

With reference to FIG. 10, the outer shape of the biological sample analysis plate 300 is 8-cm square. Three of four corners of the plate 300 are rounded, and the remaining one corner is cut off. A hole 44 is provided to make the outer shape of the plate 300 asymmetrical, whereby positions of the respective patterns can be specified. The biological sample analysis plate 300 is made of an acrylic plastic having a thickness of 2 mm. Grooves are formed on the channel formation surface, and an acrylic film having a thickness of 50 μm is adhered onto the surface, thereby producing hermetically closed channels.

Further, reference numeral 45 denotes the center of gravity of the biological sample analysis plate 300, and a hole 43 for fixing the biological sample analysis plate 300 onto the rotation part is provided around the center of gravity 45.

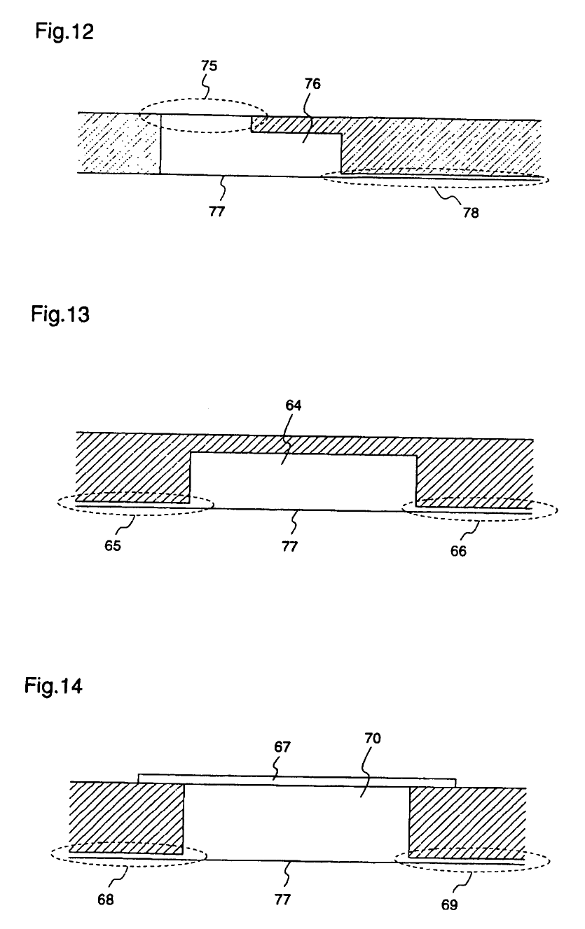

A buffer agent inlet 46 shown in FIG. 11a is an inlet for injecting the DNA conjugate as a buffer agent, a buffer agent injection part 47 is a part for temporarily holding the injected buffer agent, a sample inlet 48 is an inlet for injecting the DNA sample, and a sample injection part 49 is a part for temporarily holding the injected DNA sample. The buffer agent injection part 47 and the sample injection part 49 are identical in shape, and a cross-sectional view of the part 47 or 49 and its vicinity is shown in FIG. 12.

FIG. 12 is a cross-sectional view of the buffer agent injection part 47 or the sample injection part 49 and its vicinity, wherein hatched areas indicate the biological sample analysis plate 300. In FIG. 12, reference numeral 75 corresponds to the buffer agent inlet 46 or the sample inlet 48, and numeral 76 corresponds to the buffer agent injection part 47 or the sample injection part 49. Further, numeral 77 corresponds to the above-mentioned film, and the film 77 is adhered to the plate so as to cover the groove 76, thereby forming a closed channel 78. The channel formation surface is on the lower side.

A positive electrode insertion part 52 and a negative electrode insertion part 53 are connected through a channel 54, and further, these parts 52 and 53 are connected to the buffer agent injection part 47 through channels 50 and 51, respectively.

A chamber part 56 is connected to the sample injection part 49 by a channel 55.

Further, reference numeral 60 denotes a sample holding part, and it is connected to the chamber part 56 by channels 57 and 58. The channel 57 is narrow, and the channel 58 is wider than the channel 57. In this third embodiment, the channel 57 is 25 μm wide, while the channel 58 is 50 μm wide. Both of the channels 57 and 58 are 30 μm deep. As for the inner circumference side pattern 42′ on the biological sample analysis plate 300, the channel 57′ is 40 μm wide, the channel 58′ is 80 μm wide, and these channels are 50 μm deep.

Reference numeral 61 denotes a buffer part which is connected to the chamber part 56 by the channels 57 and 59 and to the sample holding part 60 by the channels 57 and 58. Further, a channel 62 connecting the sample injection part 49 and the buffer part 61 enables air releasing from the buffer pare 61.

The buffer agent injection part 47, the channel 50, the channel 51, the positive electrode part 52, the negative electrode part 53, and the channel 54 form a closed channel, and the buffer agent flows in the closed channel, thereby constituting a buffer agent channel 81.

With reference to FIG. 10, as shown in the inner circumference side pattern 42′, the pattern shape of the buffer agent channel is rectangle which is arranged toward the outer circumference with respect to the center of the plate 300, and the channel length L1 extending toward the outer circumference is longer than the channel length L2 extending in the rotation direction on the circumference. Thereby, a lot of channel patterns can be disposed concentrically on the plate 300.

Subsequently, the sample injection part 49, the channel 55, the chamber part 56, the channel 57, the channel 58, the channel 59, the sample holding part 60, the buffer part 61, and the channel 62 form a closed channel, and the biological sample flows in this closed channel, thereby constituting a sample channel 91. The buffer agent channel 81 and the sample channel 91 joins together at a sample quantitative sorting part 63, and the DNA sample quantified in the sample quantitative sorting part 63 is actually analyzed.

In this third embodiment, the sample channel 91 is disposed in a position inner than the buffer agent channel 81. This pattern is designed so as to narrow the spacing between the adjacent channel patterns in the concentric direction, aiming at an increase in the number of inspection items to a maximum extent. If the sample channel 91 is arranged in the concentric direction with the buffer agent channel 81 as shown in FIG. 11b, since the spacing between the adjacent channel patterns in the concentric direction is increased, the number of the channel patterns is decreased to 18 while it is 24 in this third embodiment.

The chamber part 56, the sample holding part 60, and the buffer part 61 are identical in shape, and a cross-sectional view of each part and its vicinity is shown in FIG. 13. In FIG. 13, the lower side corresponds to the channel formation surface.

In FIG. 13, a groove 64 corresponds to the chamber part 56, the sample holding part 60, or the buffer part 61, and it has a concave shape 1.5 mm deep viewed from the channel formation surface. Reference numeral 65 and 66 denotes channels extending from each part.

Next, the positive electrode part 52 and the negative electrode part 53 will be described with reference to FIG. 14.

FIG. 14 is a cross-sectional view of the positive electrode part 52 or the negative electrode part 53 and its vicinity, and the lower side corresponds to the channel formation surface. A hole 70 corresponds to the positive electrode part 52 or the negative electrode part 53, and the hole 70 penetrates through the plate 300. A film 77 and a film 67 are adhered to both surfaces, respectively, and the film 77 must be made of a nonconductive material while the film 67 may be made of a conductive material. When using a conductive material for the film 67, voltage can be applied to the sample in the electrode part 70 by applying voltage to the film 67 from the outside. When using a nonconductive material for the film 77, voltage application is achieved by puncturing the film 77 with a needle-shaped electrode and inserting it into the electrode part 70.

While the film 77 is adhered to the entire surface of the area of the plate 300 where the channels are formed, the film 67 is adhered to only an area including the electrode part and its vicinity.

Next, a description will be given of an example of specific operation until presence/absence of SNPs (Single Nucleotide Polymorphism) in the DNA sample is determined, with reference to FIG. 11a showing the specific shape of the outer circumference side pattern 42.

First of all, a DNA sample to be an analyte is prepared.

Fundamentally, DNA has a duplex-strand helical structure. In this third embodiment, however, single-strand DNA having a base length of about 60 bases including SNPs to be discriminated is prepared.

Since the methods of extraction and denaturation are not directly related to the present invention, specific description thereof will be omitted.

Next, a DNA conjugate is prepared as a buffer agent. A DNA conjugate is obtained by covalently bonding a high-molecular linear polymer to a 45′ end of single-strand DNA having a base length of 6˜12 bases. Further, the DNA conjugate has a sequence that is complemental to normal DNA but non-complemental to mutant DNA, and the bonding force of the DNA conjugate to the normal DNA is strong while the bonding force thereof to the mutant DNA is weak. Further, when performing electrophoresis, the electrophoresis speed is considerably low because the linear polymer bonded to the 45′ end acts as a weight.

It is assumed that “DNA conjugate” described hereinafter is a material containing a pH buffer agent that also serves as an electrolyte, and a DNA bonding force control agent such as MgCl₂.

When preparation of the sample is completed, the DNA conjugate and the DNA sample are injected into the plate 100. A predetermined amount of the DNA conjugate is dispensed from the buffer agent inlet 46 into the buffer agent injection part 7 using a pipeter or the like. Likewise, a predetermined amount of the DNA sample is also dispensed from the sample inlet 48 into the sample injection part 49.

Although the amount of dispensation depends on the scale of the pattern, the amount of the DNA conjugate is 3 microliters, and the amount of the DNA sample is 1 microliter.

Next, the biological sample analysis plate 300 is fixed to a motor or the like, and rotated around the center of gravity 45. At this time, the dispensed DNA conjugate and DNA sample migrate toward the outer circumference due to centrifugal force.

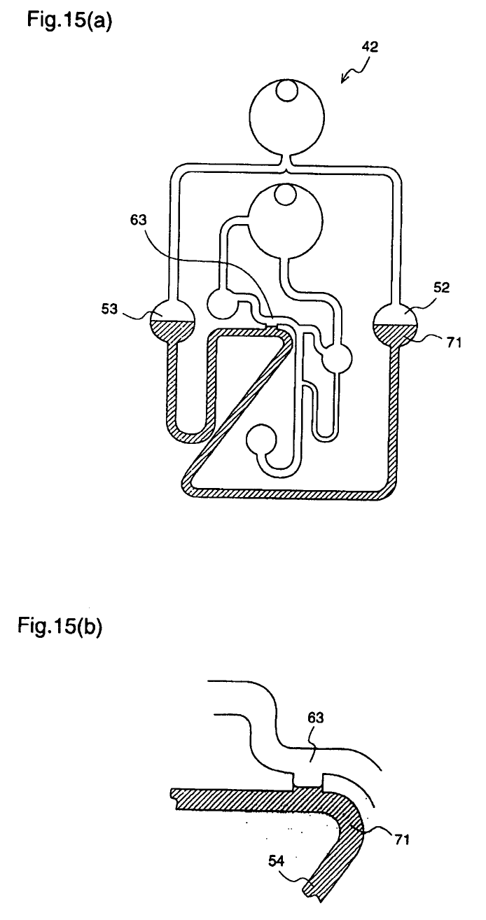

The DNA conjugate in the buffer agent injection part 47 is equally divided into the positive electrode part 52 and the negative electrode part 53 through the channel 50 and the channel 51. The DNA conjugate that has reached into the positive electrode part 52 further migrates in the channel 54a (on the right side in the figure) to reach the quantitative sorting part 63. Likewise, the DNA conjugate that has reached into the negative electrode part 53 further migrates in the channel 54b (on the left side in the figure) to reach the quantitative sorting part 63. FIG. 15(a) shows the state where migration of the DNA conjugate is stopped, two minutes after start of the rotation. Further, FIG. 15(b) is an enlarged view of the quantitative sorting part 63 and its vicinity.

The DNA conjugate 71 fills up about 70% of each of the positive electrode part 52 and the negative electrode part 53, and fully fills up the channel 54, and reaches the quantitative sorting part 63.

The fluid levels of the DNA conjugate existing in the positive electrode part 52 and the negative electrode part 53 and the fluid level of the DNA conjugate in the sample quantitative sorting part 63 are on the same circle around the center of gravity 45.

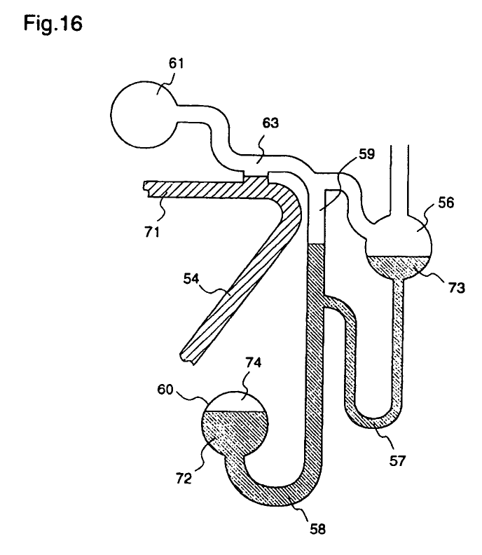

Next, migration of the injected DNA sample will be described with reference to FIGS. 11a and 16.

The DNA sample in the sample injection part 49 migrates through the channel 55, the chamber part 56, the channel 57, and the channel 58 to reach the sample holding part 60 which is positioned on the outer circumference side. However, as shown in FIG. 16, the channel 58 is connected to the outer circumference side of the sample holding part 60, and the sample holding part 60 has no hole for air releasing. Therefore, the air remaining in the sample holding part 60 is not released but compressed.

FIG. 16 shows the state of the DNA sample two minutes after start of the rotation, wherein reference numerals 72 and 73 denote the DNA sample. Reference numeral 74 denotes the compressed air, and the state where the centrifugal force and the pressure are balanced is maintained only during rotation. In this third embodiment, the rotation speed is 4000 rpm.

Next, the subsequent operation will be described.

The biological sample analysis plate 300 is rotated for a predetermined period of time, and the rotation is suddenly stopped under the state where the migrations of the DNA conjugate and the DNA sample are stopped. For example, the rotation of the plate 300 at 4000 rpm is suddenly stopped in two seconds.

Then, the DNA sample 72 existing in the sample holding part 60 starts to flow back to the channel 58 from the sample holding part 60 due to the pressure of the air 74 because the centrifugal force has gone. Further, the DNA sample migrates in the channel 57 and the channel 59 until the air 74 in the sample holding part 60 becomes the atmosphere pressure. At this time, since the channel 57 is narrower than the channel 59, the DNA sample 72 flows more into the channel 59 than into the channel 57.

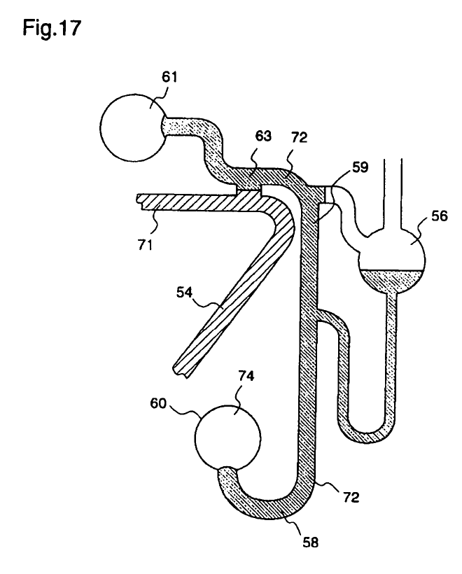

FIG. 17 shows the state immediately after the sudden stop of the plate 300.

The DNA sample 72 in the sample holding part 60 migrates through the channels 58 and 59 to reach the quantitative sorting part 63 and the buffer part 61 due to expansion of the air 74. Then, the DNA sample 72 contacts the DNA conjugate 71 that is filled in the quantitative sorting part 63. At this time, there is a certain quantity of the DNA sample 72 which passes through the channel 57 to reach the chamber part 56.

Next, the biological sample analysis plate 300 is again rotated at a medium speed for a few seconds. Thereafter, the rotation of the plate is stopped. At this time, it is important to perform deceleration moderately.

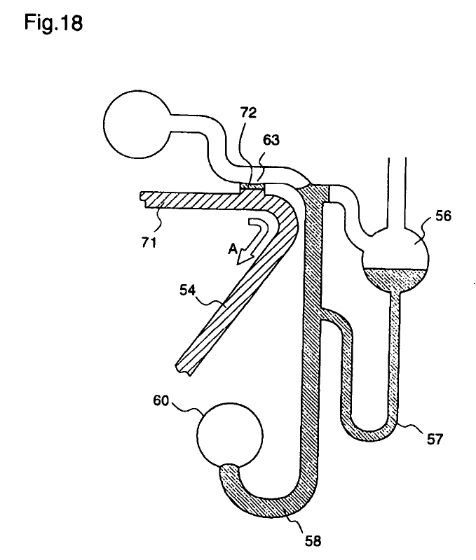

FIG. 18 shows the state where the rotation is stopped after the moderate deceleration.

At this time, the DNA sample filled in the channel 59 flows to the chamber part 56, but a slight quantity of the DNA sample 72 remains in the quantitative sorting part 63. The remaining DNA sample is separated from the other DNA sample, and therefore, it is electrically isolated from the other DNA sample.

The reason why the second rotation should be slower than the first rotation is as follows. That is, when the rotation speed is reduced to moderate the deceleration, the air 74 in the sample holding part 60 is prevented from being strongly compressed, and thereby the DNA sample is prevented from flowing back to fill the channel 59 again.

The sample DNA that remains in the quantitative sorting part 63 after the above-mentioned operation becomes the final sample to be subjected to discrimination of SNPs.

Although the inner circumference side pattern 42′ is lower in the transportability by the centrifugal force than the outer circumference side pattern 42, this difference in the transportability between these patterns due to the difference in the centrifugal force can be eliminated by making the cross-sectional area of the channel of the inner circumference side pattern 42′ larger than the cross-sectional area of the channel of the outer circumference side pattern 42. As the result, the final samples to be subjected to discrimination of SNPs can be left in the quantitative sorting parts 63 of all the patterns on the plate 41, by only one-time operation.

Next, electrophoresis of the final sample remaining in the quantitative sorting part 63 is carried out.

This electrophoresis is carried out by inserting a positive electrode and a negative electrode in the positive electrode part 52 and the negative electrode part 53, respectively, and applying a voltage of several hundreds volts to the both electrodes. Then, electrical fields occur in the channel 54 as well as in the sample quantitative sorting part 63, whereby a predetermined amount of the DNA sample 72 that remains in the quantitative sorting part 63 migrates in the channel 54 toward the positive electrode side (in the A direction in FIG. 18).

The channel 54 is filled with the DNA conjugate 71, and the DNA sample 72 performs electrophoresis while repeating bonding with the DNA conjugate 71. Since the bonding force of the normal DNA in the DNA sample with the DNA conjugate is strong as described above, the electrophoresis speed is lowered. On the other hand, since the bonding force of the mutant DNA with the DNA conjugate is weak, the electrophoresis speed is higher than that of the normal DNA. That is, when both the normal DNA and the mutant DNA exist in the DNA sample 72, the normal DNA and the mutant DNA are separated, whereby discrimination of SNPs is realized.

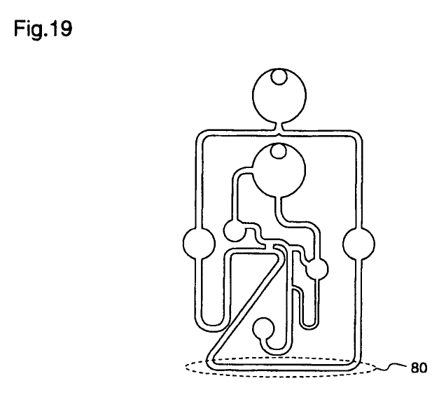

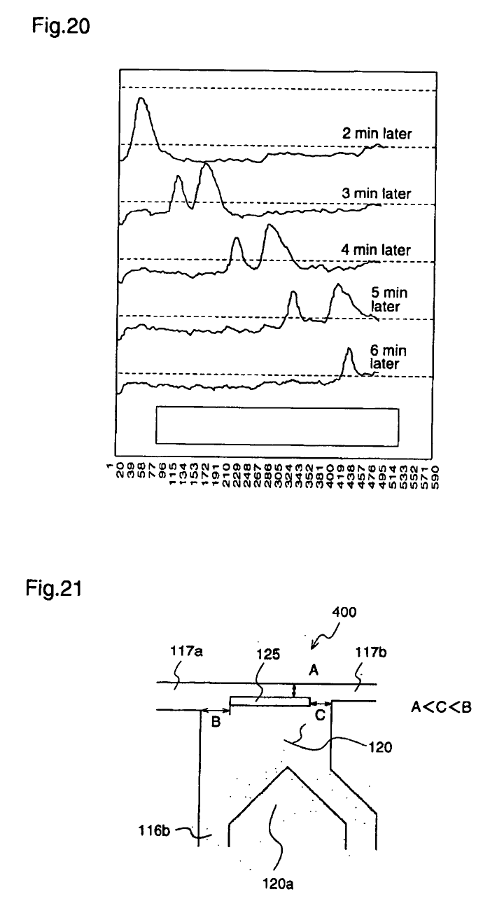

FIG. 20 is a graph obtained by scanning the DNA while the DNA performs electrophoresis in a portion 80 of the channel 54 shown in FIG. 19.

DNA detection is carried out by exciting the fluorescent-labeled (FITC) DNA with light of 470 nm, and performing photo detection in the vicinity of 520 nm. This DNA detection may be carried out by detecting absorbance at 260 nm.

The abscissa indicates the position of the portion 80, and the DNA migrates from left to right. That is, the left side is the quantitative sorting part 63, and the right side is the positive electrode part 52. The ordinate indicates the fluorescence intensity, showing the waveform that changes with time for every one minute. These waveforms show that two peaks are gradually separated. In this case, it is determined that the normal DNA and the mutant DNA of the same quantity exist in the DNA sample 72.

In the graph, the right-side peak shows the mutant DNA because the electrophoresis speed is high, while the left-side peak shows the normal DNA because the electrophoresis speed is low.

As described above, according to the biological sample analysis plate of the third embodiment, the DNA sample supply channels 55, 59, 58, and 57 and the buffer agent supply channels 50, 51, and 54 are formed such that these channels contact the quantitative sorting part 63 and the sample channels are disposed inner than the buffer agent channels, and the sample holding part 60 is provided in the DNA sample supply channel 58. The speed of the operation for rotating the biological sample analysis plate 300 is adjusted to hold a predetermined amount of the DNA sample 72 in the quantitative sorting part 63 on the plate, and the predetermined amount of the DNA sample 72 that is held is made to perform electrophoresis by the positive electrode part 52, the negative electrode part 53, and the channel 54. At this time, a difference in the electrophoresis speed is detected to detect whether the normal DNA and the mutant DNA exist in the DNA sample or not. Therefore, the channel pattern can be made compact, and plural items can be examined using one plate.

Furthermore, when the cross-sectional areas of the sample channels and the buffer agent channels are variable, two channel patterns having the same channel shape and different cross-sectional areas can be examined simultaneously, whereby inspection of more items can be carried out.

### Embodiment 4

Next, a biological sample analysis plate according to a fourth embodiment of the present invention, and a biological sample determination apparatus using the plate will be described with reference to FIGS. 21˜34.

Initially, the construction of the biological sample analysis plate 400 according to the fourth embodiment will be described with reference to FIGS. 21˜24.

In the biological sample analysis plate 400, a quantitative holding unit 120 for holding a predetermined volume of DNA sample is provided at a junction of a first channel 116 through which a supplied buffer agent flows and a second channel 117 through which a supplied DNA sample flows.

The shape of the quantitative holding part 120 and its vicinity in the biological sample analysis plate 400 will be described in detail with reference to FIG. 21.

The channel width of a sample injection channel 117a is larger than the channel width of a sample filling channel 117b. Further, an island 125 provided in the quantitative holding part 120 is a quadrangular prism having a horizontal width shorter than the width of the quantitative holding part 120 and a vertical width that is equal to or shorter than a difference between the sample injection channel 117a and the sample filling channel 117b, and a height equal to the channel depth. The island 125 is disposed between the bottom of the sample injection channel 117a and the bottom of the sample filling channel 117b so as to be separated from the left and right walls of the quantitative holding part 120.

A cross-sectional area B of an injection part from the sample injection channel 117a to the quantitative holding part 120 is equal to or larger than a cross-sectional area C of an injection part from the quantitative holding part 120 to the sample filling channel 117b. Further, a cross-sectional area A of a portion above the island 125 is smaller than the cross-sectional areas B and C.

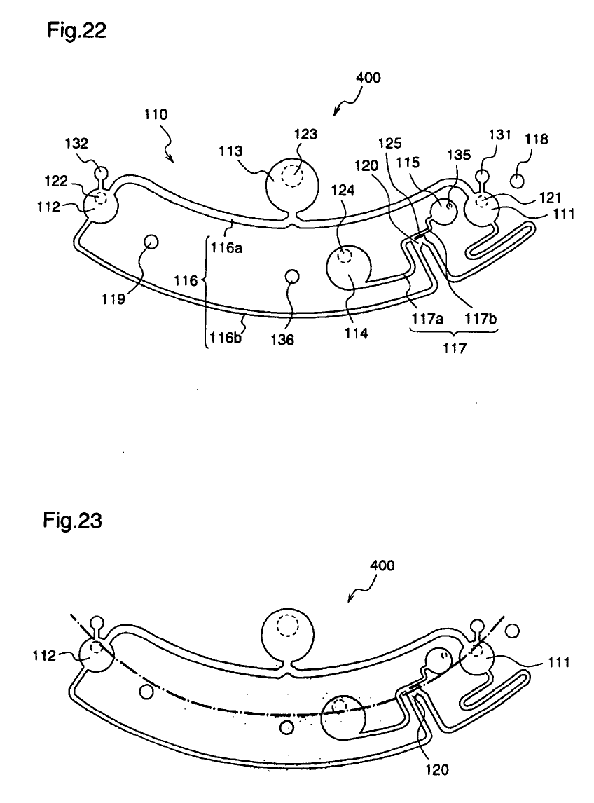

Next, the construction of the biological sample analysis plate 400 according to the fourth embodiment will be described with reference to FIGS. 22˜24. In this fourth embodiment, in order to specify the description, it is assumed that a DNA sample is used as the biological sample, and a buffer agent including a DNA conjugate for separation and a DNA bonding control agent is used as the buffer agent. Further, the biological sample analysis plate 400 adds a predetermined quantity of the DNA sample into the DNA conjugate for separation which is filled in a channel so as to make the sample perform electrophoresis, and detects fluorescence or absorbance in the channel, thereby determining presence/absence of SNPs (Single Nucleotide Polymorphism) in the DNA sample.

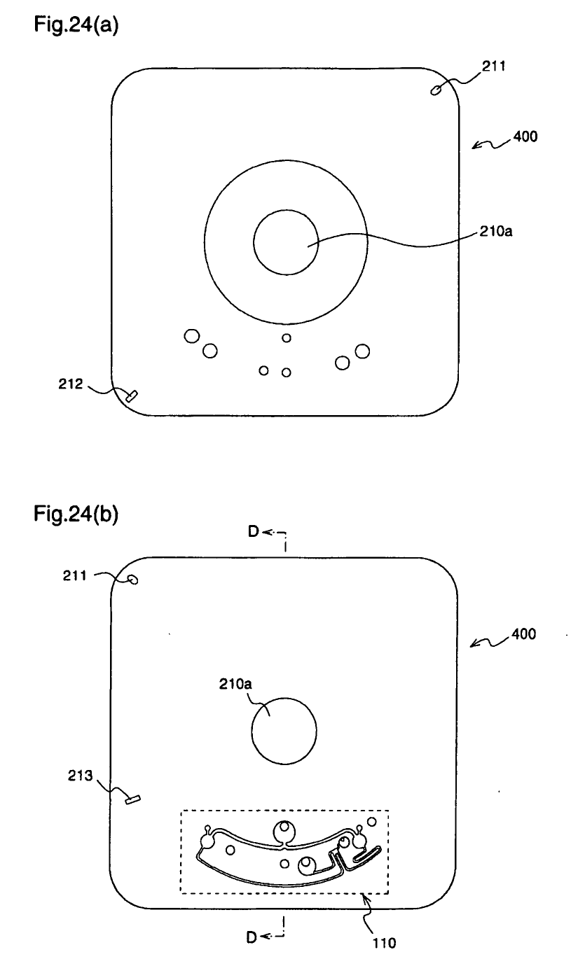

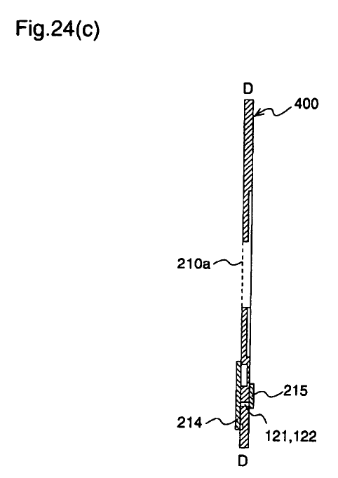

FIG. 24(a) shows a lower surface of the biological sample analysis plate 400 according to the fourth embodiment, and a specific shape of a channel patter 110 formed on the biological sample analysis plate 400. The channel pattern 110 is a minute channel which is produced by grooves having minute widths and depths, for making the biological sample perform electrophoresis.

FIG. 24(b) shows an upper surface of the biological sample analysis plate 400, and FIG. 24(c) shows a D-D cross-section of the biological sample analysis plate 400.

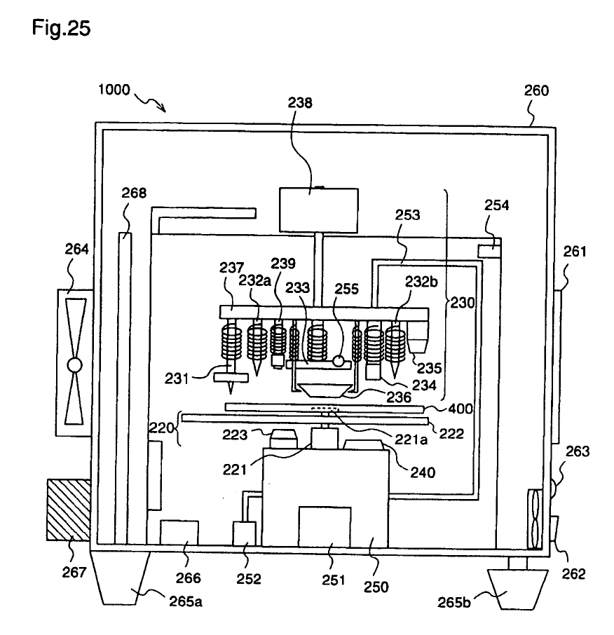

As shown in FIG. 24(a), the biological sample analysis plate 400 according to the fourth embodiment is provided with an opening 210a in its center, a fitting pin hole 211 to be fitted to a biological sample determination apparatus 1000 shown in FIG. 25, and a mark 212 for positioning.

As shown in FIG. 24(b), the channel patter 110 for migrating the DNA sample in the DNA conjugate for separation as well as a plate recognition mark 213 for detecting the biological sample analysis plate 400 with a sensor are provided at the lower surface of the biological sample analysis plate 400.

Further, as sown in FIG. 24(c), a capillary seal 214 is adhered to the lower surface of the biological sample analysis plate 400 so as to seal the channel pattern 110 formed on the biological sample analysis plate 400, and a cover film 215 is adhered to the upper surface of the biological sample analysis plate 400 so as to seal only the electrode insertion port. The capillary seal 214 is desired to be transparent because it is necessary to measure the absorbance or fluorescence of the channel by an optical detection part 240.

To be specific, as shown in FIG. 22, the channel pattern 110 comprises a first sample injection part 113 for injecting the DNA conjugate for separating the DNA sample, a second sample injection part 114 for injecting the DNA sample, a sample pool 115 to which the DNA sample injected to the second sample injection part 114 migrates, a first electrode insertion part 111 for inserting a negative electrode, a second electrode insertion part 112 for inserting a positive electrode, and a channel for connecting these parts.

The channel comprises a first channel 116 which is filled with the buffer agent, and a second channel 117 which is filled with the DNA sample. The first channel 116 and the second channel 117 join together at the quantitative holding part 120.

The first channel 116 comprises an inner circumference channel 116a which is disposed on the inner side of the plate, for connecting the first sample injection part 113 and the first electrode insertion part 111, and connecting the first sample injection part 113 and the second electrode insertion part 112, and an outer circumference channel 116b which is a channel for connecting the first electrode insertion part 111 and the second electrode insertion part 112.

The second channel 117 comprises a sample injection channel 117a for connecting the quantitative holding part 120 and the second sample injection part 114, and a sample filling channel 117b for connecting the quantitative holding part 120 and the sample pool 115.

The absorbance or fluorescence of the electrophoresis channel in a portion of the external circumference channel 116b after hydrogen-bonding reaction between the DNA sample and the DNA conjugate for separation is measured by the optical detection part 240 thereby determining presence/absence of SNPs in the quantitatively added DNA sample.

In the process of filling the quantitatively added DNA sample, if the conjugate remains in the inner circumference channel 116a of the first channel 116, electrophoresis might occur at the inner circumference channel 116a side when electrodes are inserted in the first and second electrode insertion parts 111 and 112 to apply voltage, whereby it becomes impossible to detect absorbance or fluorescence in the electrophoresis channel which is a part of the outer circumference channel 116b.

In order to prevent the DNA conjugate for separation from remaining in the inner circumference channel 116a after the process of filling the DNA conjugate, the sum of the volumes of the first sample injection part 113 and the inner circumference channel 116a should be equal to or larger than the volume of the outer circumference channel 116b, and equal to or smaller than the sum of the volumes of the first sample injection part 113, the inner circumference channel 116a, the first electrode insertion part 111, and the second electrode insertion part 112.

In order to quantify the DNA sample, as shown in FIG. 23, the quantitative holding part 120 should be positioned on the same circle as the first electrode insertion part 111 and the second electrode insertion part 112, around the center of the biological sample analysis plate 400. The reason is as follows. In the process of filling the DNA conjugate for separation, when the outer circumference channel 116b is filled with the DNA conjugate, if the position of the quantitative holding part 120 is closer to the center than the first electrode insertion part 111 and the second electrode insertion part 112, the DNA conjugate is not filled in the quantitative holding part 120. On the other hand, when the position of the quantitative holding part 120 is closer to the outer circumference than the first electrode insertion part 111 and the second electrode insertion part 112, the DNA conjugate undesirably flows into the second electrode insertion part 112 and the sample pool 115.

The quantitative holding part 120 is provided with the island 125 for suppressing migration of the biological sample into the buffer fluid. This island 125 suppresses migration of the DNA sample toward the channel 116b, whereby the DNA sample can be reliably filled in the quantitative holding part 120. Further, when the DNA sample is migrated to the sample injection part 114 by a centrifugal force with only the DNA sample in the quantitative holding part 120 contacting the first channel 116 being left, the island 125 prevents the air passing through the second channel 117 above the quantitative holding part 120 from cutting off the quantified sample, whereby a predetermined amount of DNA sample can be reliably added to the DNA conjugate for separation that is filled in the second channel 117.

Further, the bottom of the quantitative holding part 120 has a portion 120a which protrudes toward the second channel 117, and the first channel 116 is shaped such that the cross-sectional area thereof on the side where the DNA sample migrates when the DNA sample performs electrophoresis is decreased in proportion to the distance from the quantitative holding part 120. The protruding portion 120a at the bottom of the quantitative holding part 120 guides the DNA sample into the first channel 116, whereby a current is efficiently supplied to the DNA sample when a voltage is applied to the electrodes, and thus the DNA sample can perform electrophoresis reliably. Further, since the cross-sectional area of the channel in which the DNA sample migrates is decreased in proportion to the distance from the quantitative holding part 120, the migrating DNA sample is condensed to increase the concentration thereof, thereby facilitating detection of the DNA sample.

When detecting the fluorescence in the electrophoresis channel by the optical detection part 240, the fluorescent is hard to detect it the electrophoresis channel is too deep. Therefore, preferably, the channel has a large width and a shallow depth, for example, it may be 300 μm wide and 50μm deep. On the other hand, when detecting the absorbance of the electrophoresis channel by the optical detection part 240, the absorbance is hard to detect if the electrophoresis channel is too shallow. Therefore, the channel is desired to be appropriately deep, for example, it may be 300 μm wide and 300 μm deep.

Moreover, in the channel pattern 110, a first electrode standby hole 118 for keeping the electrodes 232a and 232b on standby when measurement is not carried out is provided concentrically with the first electrode insertion part 111. Further, a second electrode standby hole 119 is provided concentrically with the second electrode insertion part 112. Further, on the biological sample analysis plate 400, a pressurization standby hole 136 for keeping a pressurization part 234 on standby when the pressurization part 234 does not apply pressure is provided concentrically with the second sample inlet 114. Further, the first electrode insertion part 111 and the second electrode insertion part 112 are provided with an electrode insertion port 121 and an electrode insertion port 122 through which the electrodes 232a and 232b are inserted, and air hole 131 and an air hole 132, respectively. Further, the first sample injection part 113 is provided with a sample inlet 124, and the second sample injection part 114 is provided with a sample inlet 124, and furthermore, the sample pool 115 is provided with an air hole 135.

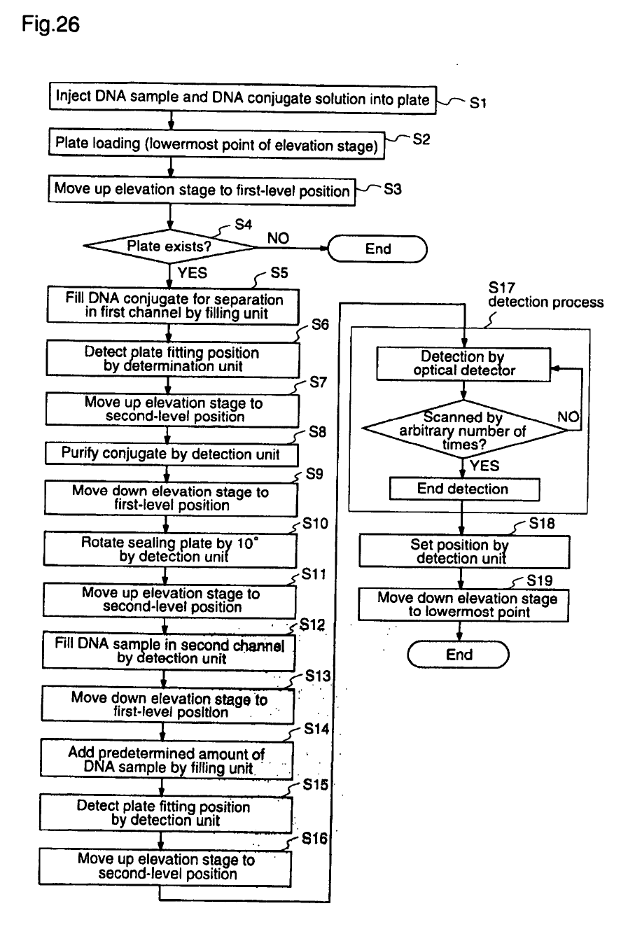

Next, the construction of the biological sample determination apparatus 1000 according to the fourth embodiment will be described with reference to FIGS. 25 and 26. FIG. 25 is a block diagram illustrating the biological sample determination apparatus 1000 according to the fourth embodiment.

With reference to FIG. 25, the biological sample determination apparatus 1000 is provided with an elevation stage 250 which includes a vertical drive motor 251 and is moved up and down by the motor 251; a filling unit 220 for filling the DNA conjugate by centrifugal force in the channels included in the pattern 110 formed on the biological sample analysis plate 210, and adding a predetermined amount of the DNA sample by centrifugal force in the channels which are filled with the DNA conjugate; an optical detection part 240 for detecting fluorescence or absorbance of the DNA sample that migrates in the DNA conjugate; a detection unit 230 for performing pressurization, heating, and voltage application on the biological sample analysis plate 210 to rotate the plate 210 with respect to the optical detection part 240; and a control substrate 268 for controlling the operation of the biological sample determination apparatus 1000.

The filling unit 220 is provided with a high-speed rotation motor 221 for rotating the biological sample analysis plate 210 at a high speed; a plate tray 222 which holds the biological sample analysis plate 210, has a portion fixed to the biological sample determination apparatus 1000, and moves from the inside to the outside of the biological sample determination apparatus 1000 or from the outside to the inside of the apparatus 1000 through a door 261 provided on a casing 260; and a plate checking sensor 223 for checking presence/absence of the plate before starting measurement. The detection unit 230 is provided with a fitting pin 231 for fixing the biological sample analysis plate 400 to the detection unit 230; two electrodes 232a and 232b for applying voltage to the channels formed on the biological sample analysis plate 400; a heater 233 for keeping the channels at a constant temperature; a pressurization part 234 for pressurizing the DNA sample injected into the channels to supply the sample into part of the channels; a positioning mark detection sensor 235 for performing positioning when the biological sample analysis plate 210 is fixed to the detection unit 230; a clamper 236 for holding the biological sample analysis plate 400; a sealing plate 237 provided with a thermistor 239 for detecting the temperature of the channels on the biological sample analysis plate 400; and a low-speed rotation motor 238 for rotating the sealing plate 237 at a low speed.

In the biological sample determination apparatus 1000 of the fourth embodiment, the elevation stage 250 is disposed in the lower part of the biological sample determination apparatus 1000, and the filling unit 220 and the optical detection part 240 are disposed on the elevation stage 250, and further, the detection unit 230 is disposed above the elevation stage 250, resulting in the more compact construction.

The clamper 236 and the heater 233 are connected to the sealing plate 237 through a damper support and a heater support, respectively. Each of the fitting pin 231, the electrode 232a, the electrode 232b, the heater 233, the pressurization part 234, the clamper 236, and the thermistor 239 which are provided on the sealing plate 237 is provided with a spring for giving an appropriate tension to the biological sample analysis plate 400, and each part is pressed against the biological sample analysis plate 400 by this spring. Further, the pressurization part 234 is connected to a pressurization pump part 252 through a pump tube 253.

The heater 233 is provided with a heater temperature detection sensor 255 for detecting the temperature of the heater 233, and an intra-apparatus temperature detection sensor 254 for detecting the temperature in the biological sample determination apparatus 1000 is provided in the apparatus 1000.

Furthermore, the biological sample determination apparatus 1000 is further provided with a pressurization pump 252 connected to the pressurization part 234 of the detection unit 230, a high-voltage power supply 266, an apparatus power supply 267, a power supply switch 262 for controlling on/off of the apparatus, an LED 263 which is turned on when the power supply switch 262 is in the ON state, a cooling fan 264 for cooling the inside of the apparatus 100, a height-adjustable rubber legs 265a and 265b for protecting the apparatus 1000 from vibration.

Hereinafter, the operation of the biological sample determination apparatus 1000 according to the fourth embodiment having the above-mentioned construction will be described with reference to FIGS. 26˜32.

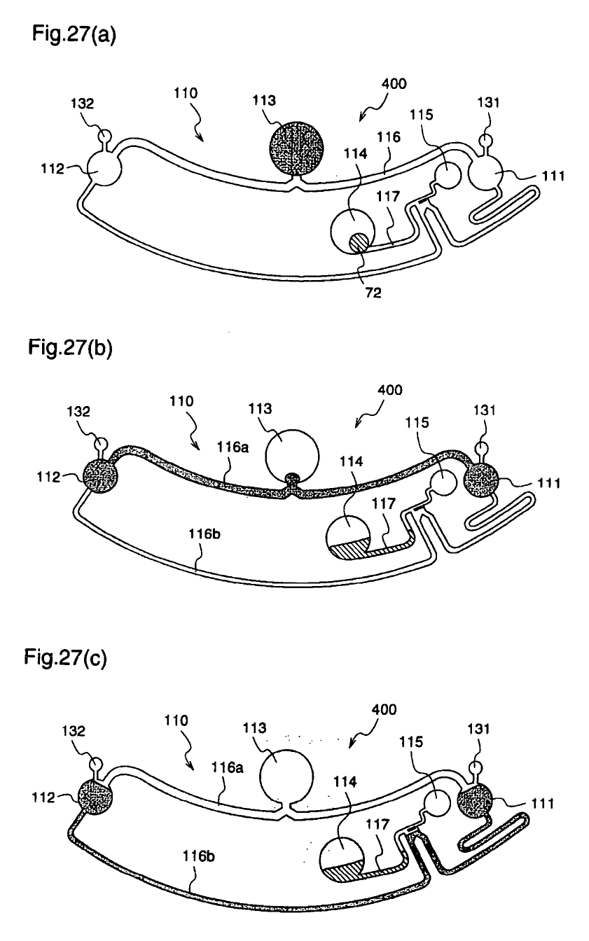

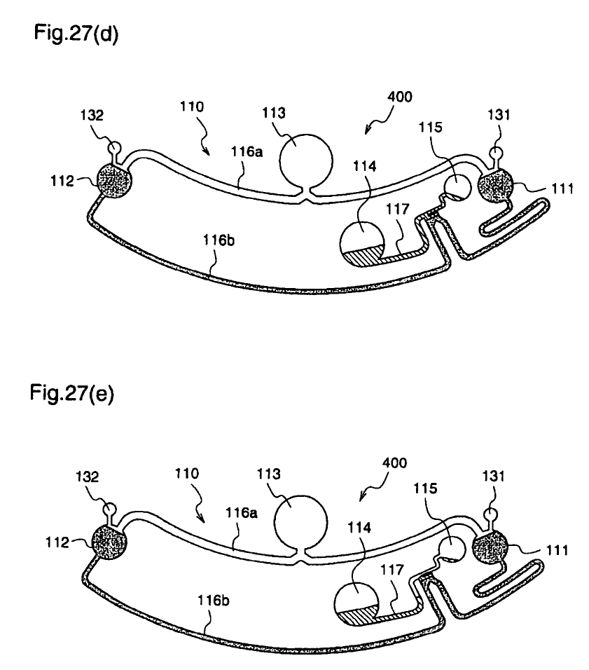

Initially, by a manual operation using a syringe, as shown in FIG. 27(a), the DNA conjugate for separation is injected from the sample inlet 123 into the first sample injection part 113 of the channel patter 110 provided on the biological sample analysis plate 400. On the other hand, the DNA sample 72 is injected from the sample inlet 124 into the second sample injection part 114 (step S1).

After the injection or the DNA conjugate for separation in step S1, plate loading is carried out. That is, the power supply switch 262 of the biological sample determination apparatus 1000 is turned ON, and a plate tray 222 is pulled out of the casing 260 through the door 261 by operating an operation button or the like (not shown), and the biological sample analysis plate 210 into which the sample is injected is set on the plate tray 222, and thereafter, the plate tray 222 is drawn into the casing 260 by operating the operation button again.

When the plate tray 222 is drawn in the casing 260 by the operation of step S2, the biological sample analysis plate 400 is automatically set in a position wherein the opening 210a of the plate 400 is fitted to the plate receiving part 221a of the high-speed rotation motor 221. It is assumed that the position of the elevation stage 250 in this state is the lowermost point (step S2).

Simultaneously with the above-mentioned loading of the biological sample analysis plate 400 into the biological sample determination apparatus 1000, the elevation stage 250 goes up from the lowermost point to the first-level position where the biological sample analysis plate 400 is held by the clamper 236, by the vertical drive motor 251 (step S3).

At this time, the optical detection part 240, the high-speed rotation motor 221, the plate checking sensor 223, and the biological sample analysis plate 400 that is fitted to the plate receiving part 221a of the high-speed rotation motor 221 also move up with the elevation stage 250, while the plate tray 222 docs not move because it is fixed to the biological sample determination apparatus 1000.

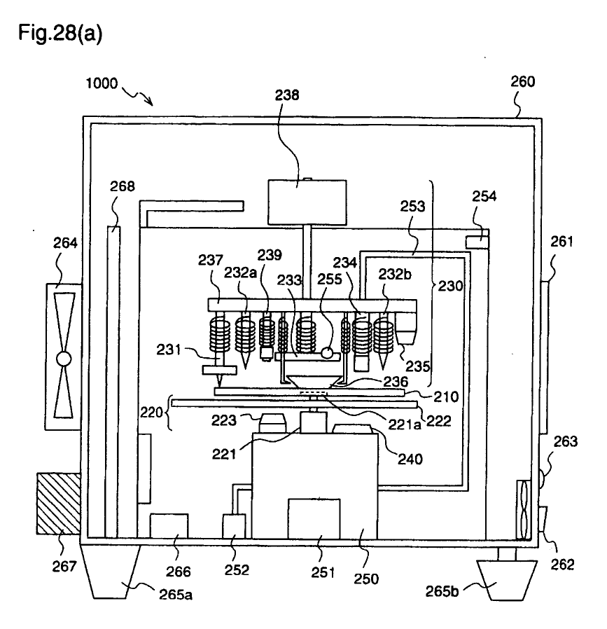

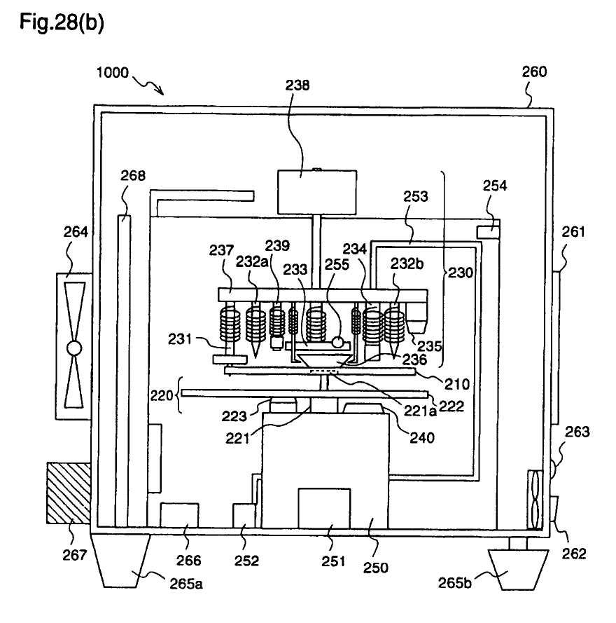

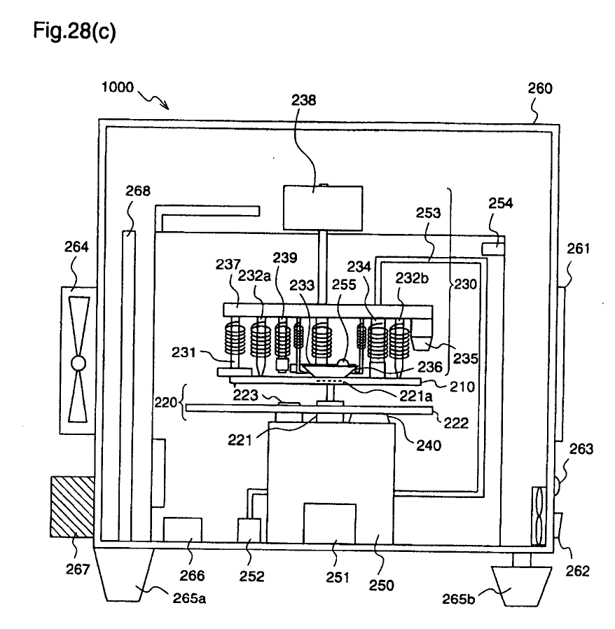

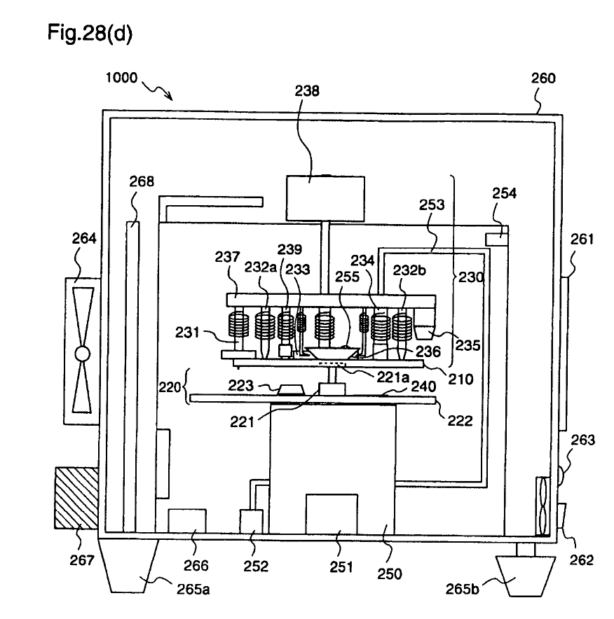

FIG. 28(a) shows the state where the elevation stage 250 goes up to the first-level position, in the biological sample determination apparatus 1000 according to the fourth embodiment.

Hereinafter, the operation until the biological sample analysis plate 400 is held by the damper 236 will be described in detail. Initially, simultaneously with loading of the biological sample analysis plate 400 in the biological sample determination apparatus 1000, the elevation stage 250 starts to go up from the lowermost point by the vertical drive motor 251, and the opening 210a of the biological sample analysis plate 400 is fitted to the plate receiving part 221a of the high-speed rotation motor 221 as shown in FIG. 25. Thereafter, the elevation stage 250 further goes up, and the biological sample analysis plate 400 also goes up together with the high-speed rotation motor 221.

When the elevation stage 250 reaches a certain position, a magnet that is disposed on the lower surface of the damper 236 that is positioned above the biological sample analysis plate 400 is connected to the plate receiving part 221a of the high-speed rotation motor 221, which is formed of a metal, whereby the biological sample analysis plate 400 is held by the clamper 236. When the plate 400 is thus connected to the plate receiving part 221a, the elevation stage 250 stops, and this stop position is the first-level position of the elevation stage 250. For example, the first-level position is 8 mm higher than the lowermost point of the elevation stage 250.

After the elevation stage 250 moves to the first-level position as described above, the process of filling the first channel 116 with the DNA conjugate for separation by centrifugal force is carried out at this first-level position by the filling unit 220. At this time, before performing the filling process, it is checked whether the biological sample analysis plate 400 is present in the determination apparatus 1000 or not (step S4˜step S5).

The checking as to whether the biological sample analysis plate 400 is present or not is carried out by detecting the plate checking mark 213 disposed on the lower surface of the biological sample analysis plate 400 using the plate checking sensor 223.

The plate checking mark 213 of the biological sample analysis plate 400 comprises a notch, a mark, or the like. For example, it is an aluminum tape in this fourth embodiment. When the biological sample analysis plate 400 is rotated by the high-speed rotation motor 221, the lower surface of the biological sample analysis plate 400 is measured with the plate checking sensor 223 which is a reflection type photosensor or the like.

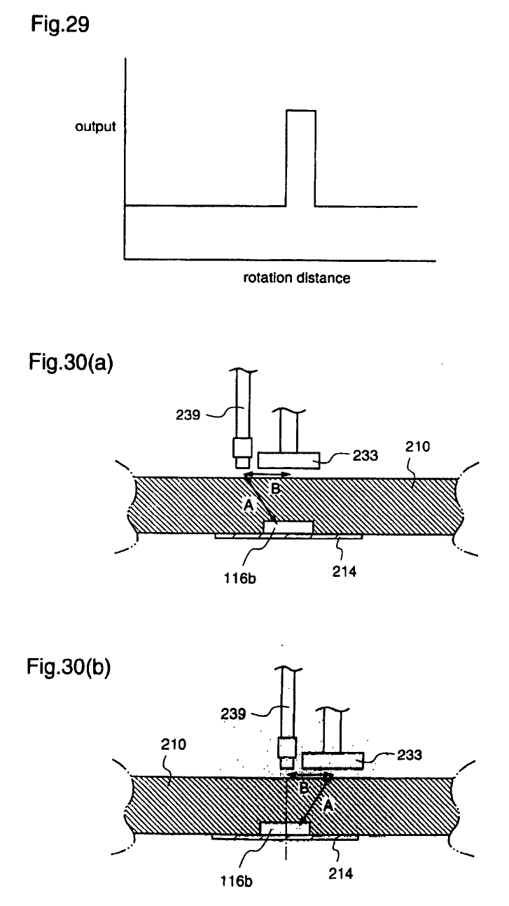

At this time, in the plate checking sensor 223, there occurs a difference in output signals between the aluminum tape as the plate checking mark 213 and a portion other than the aluminum tape as shown in FIG. 29. Therefore, it is determined that the biological sample analysis plate 400 exists when the output signal has a rising edge and a falling edge, while it is determined that no biological sample analysis plate 400 exists when the output signal has neither a rising edge nor a falling edge.

When it is determined by the plate checking sensor 223 that there is no biological sample analysis plate 400, the operation is ended at this point in time. On the other hand, when it is determined that there is a biological sample analysis plate 400, the high-speed rotation motor 221 is rotated at a predetermined rpm (about 4000 rpm in this embodiment) for two minutes, whereby the first channel 116 is filled with the DNA conjugate for separation.

Hereinafter, how the pattern 110 is filled with the DNA conjugate for separation will be described with reference to FIGS. 27(a)˜27(c)

Initially, when the biological sample analysis plate 400 is rotated at a high speed by the high-speed rotation motor 221, as shown in FIG. 27(a), the DNA conjugate for separation which is injected into the first sample injection part 113 of the pattern 110 migrates from the first sample injection part 113 through the inner circumference channel 116a of the first channel 116 to reach the first electrode insertion part 111 and the second electrode insertion part 112 as shown in FIG. 27(b) due to a centrifugal force generated by the high-speed rotation.

Further, as shown in FIG. 27(c), the DNA conjugate migrates from the first electrode insertion part 111 and the second electrode insertion part 112 through the outer circumference channel 116b of the first flow path 116, and finally, it is filled in the first electrode insertion part 111, the second electrode insertion part 112, and the outer circumference channel 116b.

On the other hand, the DNA sample injected into the second sample injection part 114 migrates in the second channel 117 along the circumference direction and is centrifugally distributed in the second channel 117 during the above-mentioned conjugate filling process by the filling unit 220. At this time, if the amount of the DNA sample injected into the second sample injection part is large, the DNA sample is undesirably filled in the second channel 117 and thereby flows into the outer circumference channel 116b by the centrifugal force that is caused by the high-speed rotation of the biological sample analysis plate 400 with the filling unit 220, before the DNA conjugate for separation is filled in the outer circumference channel 116b of the first channel 116. Accordingly, the amount of the DNA sample to be injected into the second sample injection part should be selected so that the DNA sample does not reach the outer circumference channel 116b by a centrifugal force, i.e., so that the migration of the DNA sample stops in the middle of the second channel 117 as shown in FIGS. 27(b) and 27(c).

After the respective samples are injected into the biological sample analysis plate 400 having the above-mentioned characteristics and the process of filling the conjugate by the filling unit 220 is completed in step S5, the elevation stage 250 is further moved up so as to fit the biological sample analysis plate 400 to the detection unit 230. However, in order to fit the detection unit 230 to the biological sample analysis plate 400, the fitting pin 231 of the detection unit 230 must be inserted in the fitting pin hole 211 of the biological sample analysis plate 400. Therefore, it is necessary to perform positioning of the biological sample analysis plate 400 in order to detect the position of the fitting pin hole 211 of the biological sample analysis plate 400 (step S6).

So, in this fourth embodiment, the positioning mark 212 is provided on the upper surface of the biological sample analysis plate 400 as shown in FIG. 24(a), and the positioning mark 212 is detected by the positioning mark detection sensor 235 to determine a position where the detection unit 230 is fitted to the biological sample analysis plate 400.

When the high-speed rotation motor 221 of the filling unit 220 is a servo type motor, since the servo type motor can limit the position of the biological sample analysis plate 400 after the high-speed rotation in the conjugate filling process, it is not necessary to perform positioning of the biological sample analysis plate 400.

The fitting position detection method by the detection unit 230 is identical to the operation in step S4. That is, when rotating the detection unit 230 by the low-speed rotation motor 238 which is a reflection type sensor or the like, the upper surface of the biological sample analysis plate 400 is measured by the positioning mark detection sensor 235. Then, as shown in FIG. 29, a difference in output signals occurs between the mark portion where the positioning mark 212 exists and a portion other than the mark portion, and positioning of the detection unit 230 is determined by observing a rising edge and a falling edge of the output signal. When performing positioning by rotating the detection unit 230 with the low-speed rotation motor 238, the detection unit 230 has many components disposed on the sealing plate 237, and cables and tubes (not shown) of the components are rolled up in the vicinity of the detection unit 239. Therefore, in order to prevent the cables and tubes from being tangled, the detection unit 230 is turned by half or ¾ from the right to the left to detect the position thereof, and thereafter, positioning of the detection unit 230 is performed by rotating it in the direction of less rotation angle.

After positioning of the detection unit 230, in order to fit the biological sample analysis plate 400 to the detection unit 230, the elevation stage 250 is moved up from the first-level position shown in FIG. 28(a) to the second-level position shown in FIG. 28(d) by the vertical drive motor 51 (step S7).

Hereinafter, the state until the biological sample analysis plate 400 is fitted to the detection unit 230 will be described in detail with reference to FIGS. 28(b)˜28(d).

Initially, the fitting pin 231 of the detection unit 230 starts to be inserted in the fitting pin hole 211 of the biological sample analysis plate 400 (FIG. 28(b)), and next, the electrodes 232a and 232b of the detection unit 230 are inserted in the electrode inlet 121 of the first electrode insertion part 111 and the electrode inlet 122 of the second electrode insertion part 112 of the biological sample analysis plate 400, and then the pressurization part 234 contacts the pressurization standby hole 136 of the biological sample analysis plate 400 (FIG. 28(c)). Thereafter, the elevation stage 250 goes up until the heater 233 contacts the biological sample analysis plate 400.

As the result, the filling pin 23, the electrode 232a, the electrode 232b, the pressurization part 234, the heater 233, and the thermistor 239 are pressed against the biological sample analysis plate 400, resulting in the state where the detection unit is fitted to the biological sample analysis plate 400.

Thereby, as shown in FIG. 28(d), the position of the elevation stage 250 where the detection unit 230 is fitted to the biological sample analysis plate 400 is the second-level position. For example, the second-level position of the elevation stage is 6.8 mm higher than the first-level position shown in FIG. 28(b).

After the elevation stage 250 moves up to the second-level position and the biological sample analysis plate 400 is fitted to the respective components of the detection unit 230, the temperature of the outer circumference channel 116b is measured by the thermistor 239, and the heater 233 is controlled according to the measurement result so as to keep the outer circumference channel 116b at a predetermined temperature. The reason why the outer circumference channel 116b is kept at the predetermined temperature is because, when performing detection using the optical detection part 240, it is necessary to keep the temperature condition constant. The temperature may be a constant temperature higher than room temperature.

The above-mentioned temperature is determined according to the DNA conjugate for separation to be filled and the DNA sample to be added to the DNA conjugate. For example, when the DNA sample comprises 40-60 bases while the DNA sequence of the DNA conjugate for separation, which is complementary with the target DNA to be detected, comprises 6-8 bases, the temperature is desired to be 25° C.˜45° C. The temperature of the outer circumference channel 116b is controlled by the thermistor 239 provided on the sealing plate 237.

The setting positions of the heater 233 and the thermistor 239 with respect to the outer circumference channel 116b are as follows. For example, as shown in FIG. 30(a), the heater 233 is disposed directly above the outer circumference channel 116b, and the thermistor 239 is disposed in a position beside the heater 233, wherein distances A and B shown in FIG. 30(a) are equal to each other. The heater 233 is pressed to the outer circumference channel 116b to perform heating laconically, the temperature of the biological sample analysis plate 400 is measured by the thermistor 239, and the temperature of the outer circumference channel. 116b is estimated from the measurement result to control the heater 233. Alternatively, as shown in FIG. 30(b), the heater 233 is disposed in a position beside the outer circumference channel 116b, wherein distances A and B shown in FIG. 30(b) are equal to each other, while the thermistor 239 is disposed directly above the outer circumference channel 116b, and the heater 233 is controlled while measuring the accurate temperature of the outer circumference channel 116b by the thermistor 239.

Furthermore, the rise in temperature of the heater may be measured by a heater temperature detection sensor (not shown) disposed on the heater. In this case, the amount of heat transfer from the heater 233 to the biological sample analysis plate 400 is previously measured, and a difference in temperature between the biological sample analysis plate 400 and the heater 233 is measured to control the temperature of the outer circumference channel 116b.

After the outer circumference channel 116b is set at the predetermined temperature by controlling the heater 233 as described above, the voltage supplied from the high-voltage power supply 266 is applied to the electrodes 232a and 232b disposed on the sealing plate 237, thereby performing purification of the DNA conjugate for separation.

At this time, the voltage applied to the outer circumference channel 116b is about 0.5 KV˜5 KV. Preferably, it is 1 KV˜1.5 KV. The purpose of performing purification on the DNA conjugate for separation by applying voltage to the DNA conjugate in step 57 is to remove defects (unreacted DNA and conjugate of small molecular size which are included in the DNA conjugate for separation) which occur during fabrication of the DNA conjugate for separation, thereby to obtain pure DNA conjugate for separation. The unreacted DNA and the conjugate of small molecular size which are removed at this time are transferred to and stored in the second electrode insertion part 112 into which the positive electrode is inserted.

After the conjugate purifying process is ended, pressurization is carried out by the pressurization part 234 of the detection unit 230 to fill the second channel 117 with the DNA sample (step S9˜step S12).

This pressurization is carried out as follows. The pressurization part 234 is brought into contact with the sample inlet 124 of the second sample injection part 114, and the DNA sample stored in the second sample injection part 114 is pressurized from the sample inlet 124.

In this fourth embodiment, in order to bring the pressurization part 34 to above the sample inlet 124 from above the pressurization standby hole 136 where the pressurization part 34 currently exists, the elevation stage 250 is once moved down to the first-level position from the second-level position, and the detection unit 230 is slightly rotated by the low-speed rotation motor 238 (about 10 degrees), and thereafter, the elevation stage 250 is moved up to the second-level position. Thereby, when the elevation stage 250 is moved up to the second-level position to perform pressurization, the pressurization part 234 can be brought into contact with the sample inlet 124 of the second sample injection part 114. At this time, since the electrodes 232a and 232b are also rotated simultaneously, the electrode 232a and the electrode 232b are inserted in the first electrode standby hole 118 and the second electrode standby hole 119 which are disposed concentrically with the first electrode insertion part 111 and the second electrode insertion part 112, respectively, thereby to keep the electrodes on standby.

In the state where the electrode 232a and the electrode 232b stand by in the first electrode standby hole 118 and the second electrode standby hole 119, respectively, the second sample injection part 114 in which the DNA sample is stored is pressurized by the pressurization part 234.

By this pressurization, the DNA sample which has been centrifugally distributed in only the circumference direction of the second channel 117 from the second electrode insertion part 112 as shown in FIGS. 27(b) and 27(c), can be migrated radially in the sample injection channel 117a of the second channel 117 as shown in FIG. 27(d), and a part of the DNA sample thus migrated is migrated through the sample filling channel 117b to the sample pool 115, whereby the second flow channel 117 can be filled with the DNA sample.

After performing the pressurization in step S12, the elevation stage 250 is moved down from the second-level position to the first-level position by the vertical drive motor 251 (step S13), and a predetermined amount of DNA sample is supplied to a part of the outer circumference channel 116b which is filled with the DNA conjugate for separation, by centrifugal force, using the filling unit 220 (step S14).

To be specific, the biological sample analysis plate 400 is rotated at a predetermined rpm (about 4000 rpm in this embodiment) for about 10 seconds by the high-speed rotation motor 221, whereby the DNA sample which is filled in the second channel 117 between the second sample injection part 114 and the sample pool 115 as shown in FIG. 27(d) is distributed by centrifugal force caused by the high-speed rotation so as to leave only a portion of the DNA sample which contacts the DNA conjugate for separation filled in the outer circumference channel 116b as shown in FIG. 27(e).

At this time, the DNA sample filled in the second channel 117 is quantified by the height of the protruding part 120a at the bottom of the sample injection channel in the quantitative holding part 120. Further, the island 125 is provided between the sample injection channel 117a and the sample filling channel 117b, and the above-mentioned relationship of the cross-sectional areas A, B, and C, i.e., A<C<B, is employed. Thereby, since the air passage is restricted to only the portion above the island 125 when the DNA sample is migrated in the circumference direction of the second channel 117 by centrifugal force, the DNA sample that contacts the DNA conjugate for separation filled in the outer circumference channel 116b is prevented from being cut off.

The above-mentioned construction enables application of a predetermined amount of DNA sample to the DNA conjugate for separation that is filled in the outer circumference channel 116b. During the high-speed rotation, the DNA conjugate for separation does not scatter from the cover film 215 that is torn off in the conjugate purifying process in step S8. The reason is as follows. Since the DNA conjugate for separation at this point in time is filled in only outer-circumference side portions of the first electrode insertion part 111 and the second electrode insertion part 112 as shown in FIG. 27(d), even when the biological sample analysis plate 400 is rotated at a high speed by the high-speed rotation motor 221 in this state, the DNA conjugate for separation does not migrate due to a centrifugal force.

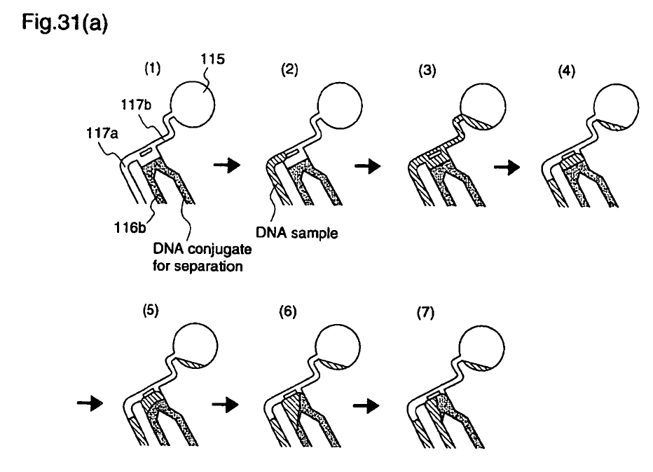

The migration states of the DNA sample described above are shown in (1)˜(4) in FIG. 31. FIG. 31-(1) shows the state where filling of the DNA conjugate for separation is ended, in which no DNA sample exists in the area filled with the DNA conjugate, migration of the DNA sample is stopped in the middle of the second channel. 117, and the DNA sample is centrifugal-distributed to the second channel.

Thereafter, when pressurization is performed by the pressurization part 234, the DNA sample that is centrifugal-distributed in the second channel 117 as shown in FIG. 31-(2) migrates into the sample pool 115 as shown in FIG. 31-(3), whereby the DNA sample is filled in the second channel 117.

Then, the biological sample analysis plate 400 is rotated at a high speed by the high-speed rotation motor 221, whereby, as shown in FIG. 31-(4), a predetermined amount of the DNA sample is added into the quantitative holding part 120 such that the DNA sample contacts the DNA conjugate for separation in parallel.

Thereafter, in order to perform measurement by the detection unit 230, the elevation stage 250 must be moved up from the first-level position to the second-level position by the vertical drive motor 251. At this time, like the process in step S7, the positioning mark 212 provided on the upper surface of the biological sample analysis plate 400 is detected by the positioning mark detection sensor 235, thereby performing positioning of the biological sample analysis plate 400 (step S15). Since the specific operation for the positioning is identical to the operation in step S6, repeated description is not necessary.

After the positioning of the biological sample analysis plate 400, the elevation stage 250 is moved up to the second-level position by the vertical drive motor 251, and the fitting pin 231 of the sealing plate 237 is inserted in the fitting pin hole 211 of the biological sample analysis plate 400 to fit the sealing plate 237 to the biological sample analysis plate 400. Thereafter, the outer circumference channel 116b is heated to a predetermined temperature by the heater 233, and the voltage supplied from the high-voltage power supply 266 is applied to the electrodes 232a and 232b to make the DNA sample perform electrophoresis in the outer circumference channel 116b that is filled with the DNA conjugate for separation, and fluorescence to absorbance of the outer circumference channel 116b is detected by the optical detection unit 240 (step S16˜step S17).

The states of the DNA sample are shown in (5)˜(7) in FIG. 31. FIGS. 31-(5)˜31-(7) show the states where voltage application is started and the DNA sample performs electrophoresis in the DNA conjugate for separation that is filled in the outer circumference channel 116b. As described above, even when the DNA sample just contacts the DNA conjugate in parallel with the conjugate without mixing the DNA sample into the DNA conjugate by agitation, it is possible to make the DNA sample migrate in the DNA conjugate by electrophoresis by only applying a voltage to the outer circumference channel 116b.

Since the bottom of the quantitative holding part 120 has the protruding portion 120a, current easily flows into the DNA sample when voltage is applied, whereby electrophoresis of the DNA sample is facilitated. Further, when the DNA sample migrates from the quantitative holding part 120 to the electrophoresis channel, since the cross-sectional area of the electrophoresis channel through which the DNA sample migrates by electrophoresis decreases in proportion to the distance from the quantitative holding part 120, the concentration of the DNA sample is increased by the effect of aggregation due to the channel shape in the process of migration of the DNA sample to the electrophoresis channel.

Thereby, the absorbance or fluorescence of the channel to be detected is increased, and detection by the optical detection part 240 is facilitated.

Detection of absorbance or fluorescence by the optical detection part 240 is carried out as follows. That is, the biological sample analysis plate 400 which is fitted to the sealing plate 237 is rotated by the low-speed rotation motor 238 with respect to the optical detection part 240 disposed above the elevation stage 250, and the absorbance or fluorescence of the electrophoresis channel portion of the outer circumference channel 116b is detected at predetermined intervals, for example, every one minute from start of the measurement. In order to indicate that measurement is being carried out, the second LED (not shown) is turned on simultaneously with voltage application to the electrodes 232a and 232b, whereby the door 261 of the biological sample determination apparatus 1000 is prevented from being opened by mistake during the measurement.

More specifically, the fitted biological sample analysis plate 400 and sealing plate 237 are rotated about one time by the low-speed rotation motor 238, and thereafter, these plates are rotated backward to return to the initial position. This operation is repeated to make the optical detection part 240 scan the electrophoresis channel of the outer circumference channel 116b in the pattern 110, thereby measuring absorbance or fluorescence of the electrophoresis channel. The scanning of the electrophoresis channel C by the optical detection part 240 is performed only in the direction along which the DNA sample performs electrophoresis, and no scanning is carried out in the opposite direction.

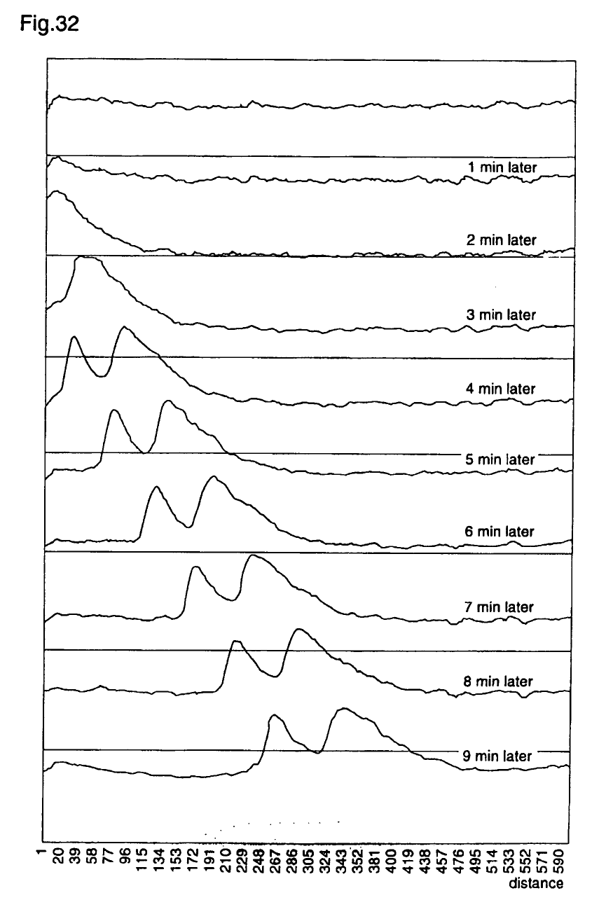

FIG. 32 is a diagram illustrating the result of detection when absorbance of the electrophoresis channel is detected using the biological sample determination apparatus 1000 according to the fourth embodiment, wherein the abscissa shows the distance of the outer circumference channel, and the ordinate shows the absorbance. In FIG. 32, a solution obtained by mixing a solution in which 0.5 mM of magnesium chloride as a DNA bonding control agent is added to 10 mM of Tris-Borate buffer solution, and a DNA conjugate for separation (conjugate 5′-TAACGGT—3-) is filled in the outer circumference channel 132, and a labeled DNA sample including mutant DNA (5′-ATGTGGAACCTTTACTAAAG-3′) and wild DNA (5′-ATGTGGAACCGTTACTAAAG-3′) is injected into the outer circumference channel 132, thereby to make the DNA sample perform electrophoresis in the electrophoresis channel C in the outer circumference channel 116b.

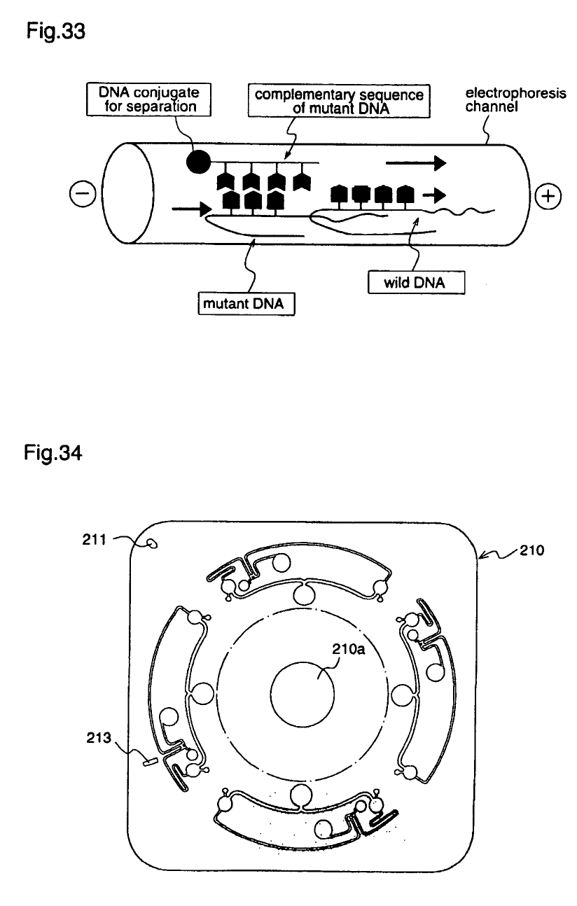

When mutant DNA is included in the DNA sample, this mutant DNA is trapped by the DNA conjugate for separation having a sequence that is complementary with the mutant DNA as shown in FIG. 33, whereby the electrophoresis speed of the mutant DNA is lower than that of the wild DNA. As the result, the wild DNA and the mutant DNA are separated as shown in FIG. 32 when absorbance of the electrophoresis channel C is detected by the optical detection part 240, and two peaks of absorbance appear. On the other hand, when no mutant DNA is included in the DNA sample, only one peak of absorbance appears. Thereby, it becomes possible to determine as to whether mutant DNA is included in the DNA sample or not.

It is also possible to measure absorbance at a certain point in the electrophoresis channel C in the outer circumference channel 116b, without rotating the biological sample analysis plate 400 by the low-speed rotation motor 238. In this fourth embodiment, however, absorbance is not measured at a certain point in the electrophoresis channel C, but the biological sample analysis plate 400 is rotated at a low speed by the low-speed rotation motor 238, and the entire electrophoresis channel C is scanned with the optical detection part 240 to measure absorbance of the entire electrophoresis channel C at predetermined time intervals.

The reason is as follows. For example, the DNA sample to be measured has a difference in speeds between the wild DNA and the mutant DNA which are included in the DNA sample, in a position where absorbance is not measured, while it has no difference in speeds in a position where absorbance is measured. Therefore, accurate results can always be obtained by scanning the entire electrophoresis channel C by the optical detection part 240 at predetermined intervals to measure fluorescence or absorbance.

When an arbitrary number of times of scanning (nine times in this fourth embodiment) on the outer circumference channel 116b by the optical detection part 240 are ended, voltage application to the electrodes 232a and 232b by the high-voltage power supply 266 is stopped, and heating by the heater 233 is also stopped. Then, the biological sample analysis plate 400 as well as the sealing plate 237 are rotated by the low-speed rotation motor 238 so that the biological sample analysis plate 400 is positioned above the plate tray 222, thereby setting the position of the plate 400 (step S18).

After the position setting, the elevation stage 250 is moved down from the second-level position to the first-level position by the vertical drive motor 251, and the damper 236 and the plate receiving part 221a are separated in the first-level position, and thereafter, the elevation stage 250 is further moved down to the lowermost position (step S19), whereby the biological sample analysis plate 400 is held on the plate tray 222. At this time, the biological sample analysis plate 400 can be discharged from the biological sample determination apparatus 1000.

As described above, according to the biological sample analysis plate 400 and the biological sample determination apparatus 1000 according to the fourth embodiment, when the DNA conjugate is filled in the biological sample analysis plate, the biological sample analysis plate 400 is provided with the quantitative holding part for holding a predetermined volume of biological sample, at a junction of the channel through which the buffer agent flows and the channel through which the biological sample flows, and the quantitative holding part is provided with the means for suppressing the flow of the held biological sample into the buffer channel. The DNA conjugate is filled in the outer circumference channel 116b of the biological sample analysis plate 400 by the filling unit 220 using centrifugal force, and the DNA sample is pressurized and a predetermined amount of the DNA Sample is added to the DNA conjugate for separation using centrifugal force, and thereafter, voltage is applied to the electrodes 232a and 232b provided in the detection unit 230 to make the DNA sample perform electrophoresis. Then, the biological sample analysis plate 400 is rotated at a low-speed by a few times for every predetermined interval using the detection unit 230, and the entire electrophoresis channel portion of the outer circumference channel 116b is scanned by a predetermined number of times using the detection unit 230 and the optical detection part 240, thereby to measure absorbance or fluorescence in the electrophoresis channel portion. Therefore, existence of target DNA to be detected, which is included in the DNA sample obtained by extracting specific DNA from cell or blood, can be determined accurately in short time, without using a capillary tube that needs complicated preparation work, whereby discrimination of various diseases or research of DNA abnormality can be carried out accurately and speedily.

Further, in the biological sample analysis plate 400 according to the fourth embodiment, the quantitative holding part 120 is provided at the junction of the channel through which the buffer agent flows and the channel through which the biological sample flows, and the quantitative holding part is provided with the island so that the cross-sectional areas of the respective channels have the relationship of A<C<B as described above. Therefore, when the DNA sample migrates, the air passage is restricted to only a portion above the island 125, whereby the DNA sample that contacts the DNA conjugate for separation is not cut out, resulting in accurate analysis.

While in this fourth embodiment the biological sample is a DNA sample and it is determined whether a mutant sample is included in the DNA sample or not, the present invention is not restricted to such usage, and it can be applied to antigen-antibody reaction or enzyme reaction.

Further, while in this fourth embodiment only one pattern 110 is formed on the biological sample analysis plate 400, four identical patterns may be formed on the biological sample analysis plate 210 as shown in FIG. 34. In this use, it is necessary to provide four sets of the electrode 232a, the electrode 232b, the heater 233, the pressurization part 234, and the thermistor 239 which are disposed on the sealing plate 237. Further, in the case where four patterns are provided on the biological sample analysis plate 400, when performing measurement by the detection unit 230, application of voltage to the electrodes 232a and 232b to be inserted in the respective patterns by the high-voltage power supply should be shifted for each pattern, considering the time lag in measurement for each pattern. Thereby, the electrophoresis time of data during measurement can be made equal in the respective patterns. The number of times the measurement is repeated can be set according to user requests.

Further, when plural patterns are formed on the biological sample analysis plate 400 as shown in FIG. 34, it is possible to use the respective channels not only for discrimination of SNPs of one person but also for discrimination of SNPs of a plurality of different persons. For example, the same DNA conjugate is filled in all the patterns, and DNA samples of different persons as many as the number of patterns formed on the biological sample analysis plate are injected into the respective patterns, whereby discrimination of the same SNP can be performed on the plural persons by one-time measurement, and data relating to the distribution conditions for the respective SNPs can be obtained at one time.

### Embodiment 5

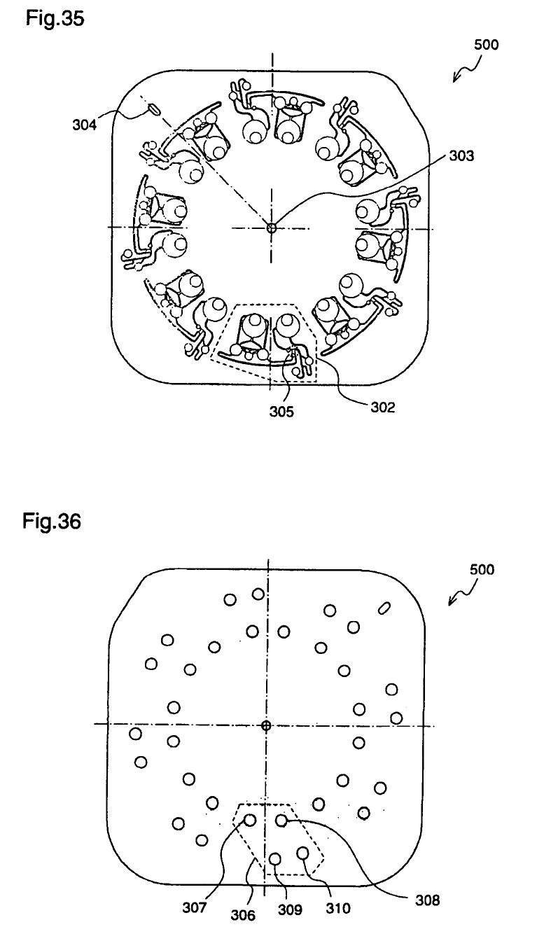

Next, a biological sample analysis plate according to a fifth embodiment of the present invention will be described with reference to FIGS. 35˜41.

According to the present invention, a biological sample is migrated in a buffer agent to carry out biological, enzymatical, immunological, and chemical reactions, thereby to analyze the biological sample easily, inexpensively, accurately, and speedily.

In this fifth embodiment, in order to specify the description, it is assumed that the biological sample is a DNA sample, and the buffer agent includes a DNA conjugate for separation and a DNA bonding control agent. The biological sample analysis plate adds a predetermined quantity of the DNA sample into the DNA conjugate for separation which is filled in channels to make the sample perform electrophoresis, and detects fluorescence or absorbance in the channels, thereby to determine presence/absence of SNPs (single Nucleotide Polymorphism) in the DNA sample.

Initially, the construction of the biological sample analysis plate 500 according to the fifth embodiment will be described with reference to FIGS. 35˜39.

FIG. 35 is a diagram illustrating the biological sample analysis plate 500 viewed from a channel formation surface thereof, according to the fifth embodiment. FIG. 36 is a diagram illustrating the biological sample analysis plate 500 viewed from a sample injection surface thereof.

The outer shape of the biological sample analysis plate 500 is 8-cm square. Three of four corners are rounded, and the remaining one corner is different in shape from the other three corners. Further, holes 3 and 4 are provided to make the outer shape of the plate 500 asymmetrical, whereby positions of the channel patterns can be specified. The biological sample analysis plate 500 is made of an acrylic plastic, and it is 40 mm square in outer shape and is 2 mm thick. Grooves are formed on the channel formation surface, and an acrylic film having a thickness of 50 μm is adhered onto the surface, thereby producing hermetically closed channels.

Injection of the samples into the biological sample analysis plate 500 is carried out from a DNA inlet 307 and a buffer inlet 308 using a pipeter or the like. A voltage application part 309 and a voltage application part 310 are parts to which voltage for making the DNA sample perform electrophoresis is applied.

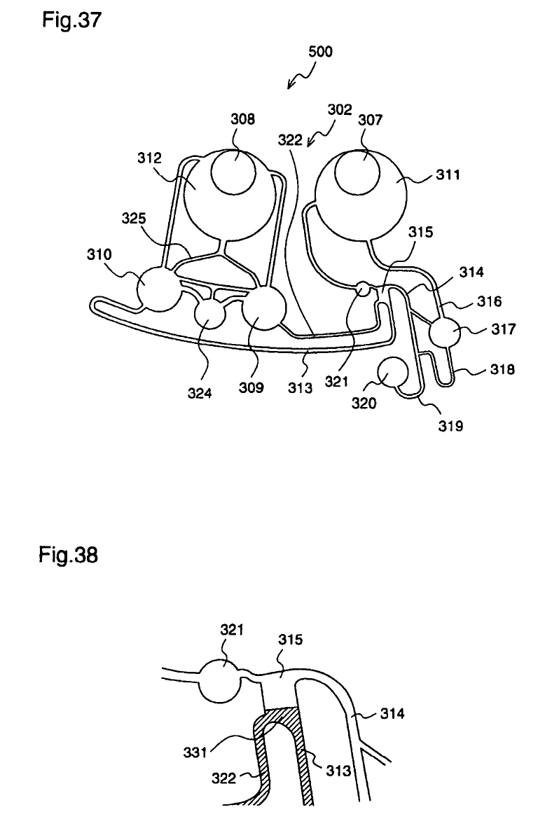

FIG. 37 is a diagram illustrating a specific shape of a channel pattern 302 formed on the biological sample analysis plate 500 shown in FIG. 35. In FIG. 37, the pattern 302 comprises minute channels which are formed of grooves having minute widths and depths, to be used for analysis of the biological sample. On the biological sample analysis plate 500, eight pieces of channel patterns 302 shown in FIG. 37 are formed radially, whereby DNA analysis for 8 analytes can be simultaneously carried out.

The channels according to this fifth embodiment are 50 μm deep, and 100-300 μm wide.

However, a sample injection part 311, a buffer injection part 312, a sample chamber 317, a sample holding part 320, a buffer part 321, and an overflow part 324 are 1.5 mm deep, and the sample inlet 307 and the buffer inlet 308 penetrate the plate 500 so that the respective samples can be injected from the sample injection surface. Further, a negative electrode part 309 arid a positive electrode part 310 also penetrate the plate 500 so that voltage can be applied from the sample injection surface.

Next, the channel pattern 302 shown in FIG. 37 will be described.

The positive electrode part 310 is an insertion part for a positive electrode, and the negative electrode part 309 is an insertion part for a negative electrode. These electrode parts are connected to each other through a channel 313, a channel 322, and a sample quantitation part 315. Further, the respective electrode parts 310 and 309 are connected to the buffer injection part 312 through a channel 325. Further, when the buffer agent supplied from the buffer injection part 312 exceeds a predetermined value in the positive electrode part 310 and the negative electrode part 309, the overflow part 324 discharges the excess amount of buffer agent by overflow.

The sample holding unit 320 is connected to the sample chamber 317 through a channel 318 and a channel 319 which are components of a sample channel (fifth channel). The channel 318 is narrow, and the channel 319 is broader than the channel 318. In this fifth embodiment, the channel 318 is 100 μm wide while the channel 319 is 200 μm wide.

The buffer part 321 is connected to the sample chamber 317 and the sample holding part 320 through the channel 314, the channel 318, and the channel 319.

The sample quantitation part 315 is disposed at a junction of the channel 313 and the channel 322 which are components of a buffer channel (sixth channel), and quantifies the DNA sample.

The DNA sample quantified by the sample quantitation part 315 migrates in the channel 313 filled with the buffer agent, by applying a voltage across the negative electrode part 309 and the positive electrode part 310.

As for the relationship between the sample quantitation part 315 and the channel 313, the channel width is gradually decreased from the sample quantitation part 315 to the channel 313, whereby the DNA sample is condensed when it performs electrophoresis.

The condensed DNA sample is supplied to the channel 313, and there occurs a difference in electrophoresis speeds between normal DNA and mutant DNA due to a difference in bonding forces thereof to the buffer agent, resulting in highly sensitive discrimination of SNPs.

As for the voltage applying method, a conductive film is adhered to the sample injection surface, and negative voltage and positive voltage are applied to the negative electrode part 309 and the positive electrode part 310, respectively. Alternatively, a film comprising acrylic or PET is adhered to the sample injection surface, and needle type electrodes each having a sharp tip are inserted through the film to apply voltage.

Hereinafter, examples of specific operation and action until determination of presence/absence of SNPs (Single Nucleotide Polymorphism) in the DNA sample will be described with reference to FIG. 37.

Initially, a DNA sample to be an analyte is prepared.

Fundamentally, DNA has a duplex-strand helical structure. In this fifth embodiment, however, single-strand DNA having a base length of about 60 bases including SNPs to be discriminated is prepared.

Since the method of extraction and denaturation is not directly related to the present invention, specific description thereof will be omitted.

Next, a DNA conjugate is prepared as a buffer agent. A DNA conjugate is obtained by covalently bonding a high-molecular linear polymer to a 5′ end of single-strand DNA having a base length of 6-12 bases. Further, the DNA conjugate has a sequence that is complementary to normal DNA but non-complementary to mutant DNA, and the bonding force of the DNA conjugate to the normal DNA is strong while the bonding force thereof to the mutant DNA is weak. Further, in the case of performing electrophoresis, the electrophoresis speed is considerably low because the linear polymer bonded to the 5′ end acts as a weight.

It is assumed that “DNA conjugate” described hereinafter is a material containing a pH buffer agent that also serves as an electrolyte, and a DNA bonding force control agent such as MgCl₂.

When preparation of the samples is completed, the DNA conjugate and the DNA sample are injected into the plate 301. A predetermined amount of the DNA conjugate is dispensed from the buffer agent inlet 308 into the buffer agent injection part 312 using a pipeter or the like. Likewise, a predetermined amount of the DNA sample is also dispensed from the sample inlet 307 into the sample injection part 311.

Although the amounts of the samples to be dispensed depend on the scale of the pattern, in this fifth embodiment, the amount of the DNA conjugate is 18 microliters, and the amount of the DNA sample is 2 microliters.

Next, the biological sample analysis plate 301 is fixed to a motor or the like, and rotated around a hole 303 that is the gravity center of the plate. At this time, the dispensed DNA conjugate and DNA sample migrate toward the outer circumference due to centrifugal force.

The DNA conjugate in the buffer agent injection part 312 is equally divided to the positive electrode part 310 and the negative electrode part 309 by the channel 325. The DNA conjugate that has reached into the positive electrode part 310 further migrates through the channel 313 to reach the sample quantitation part 315. Likewise, the DNA conjugate that has reached into the negative electrode part 309 further migrates through the channel 322 to reach the sample quantitation part 315. FIG. 38 shows the state of the sample quantitation part 315 and its vicinity, two minutes after the start of rotation.

The DNA conjugate 331 is filled in the positive electrode part 310 and the negative electrode part 309 up to about 70%, respectively, and fills up the channel 313 and the channel 322, although not shown in the figure.

At this time, the DNA sample exists in the sample chamber 317, the sample holding part 320, and the channels connecting them. The DNA sample is injected into the sample holding part 320 by centrifugal force, and further, it is maintained at a pressure higher than the atmosphere pressure because there is no channel or hole for air releasing.

Then, the rotation is suddenly stopped. For example, the rotation at 4000 rpm is brought to stop in two seconds.

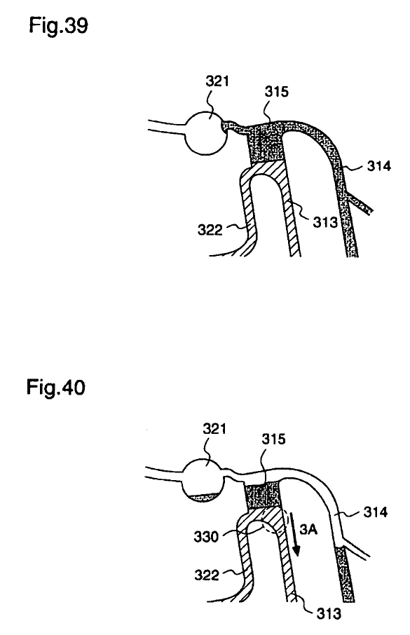

Subsequently, as shown in FIG. 39, the DNA sample that has been stored in the sample holding part 60 flows back to the channel 319 until the pressure in the sample holding part 320 is reduced to the atmosphere pressure because the centrifugal force has gone, and part of the DNA sample reaches to the sample chamber 311 while the other reaches the sample quantitation part 315.

Then, the DNA sample contacts the DNA conjugate in the sample quantitation part 315. At this time, a quantity of the DNA sample migrates Lo the buffer part 321.

Next, the biological sample analysis plate 500 is again rotated at a medium speed for a few seconds. When the rotation is stopped, it is important to perform deceleration moderately. FIG. 40 shows the state after stopping.

While a predetermined amount of the DNA sample remains in the sample quantitation part 315, the surrounding excess DNA sample flows back to the sample holding part 320 and the sample chamber 317. However, even when the rotation is stopped, since the rotation speed is low and the deceleration is moderate, the DNA sample never flows back to the sample quantitation part 315.

The DNA sample remaining in the sample quantitation part 315 is electrically isolated from the other DNA sample.

Through the above-mentioned operation, the sample DNA remaining in the sample quantitation part 315 becomes the final sample to be subjected to discrimination of SNPs.

Next, electrophoresis is carried out as follows. A positive electrode and a negative electrode are inserted in the positive electrode part 310 and the negative electrode part 309, respectively, and voltage of several hundreds volts is applied to the both electrodes. Then, electrical fields occur in the channels 313 and 322, and further, in the sample quantitation part 315, whereby the DNA sample remaining in the sample quantitation part 315 migrates in the channel 313 toward the positive electrode side (in the 3A direction in FIG. 40).

At this time, as shown in the condensation part 330 In FIG. 40, the channel width is gradually decreased from the sample quantitation part 315 to the channel 313. That is, the cross-sectional area of the channel is decreased from the sample quantitation part 315 toward the channel 313. In this condensation part 330, the DNA sample migrates while being condensed.

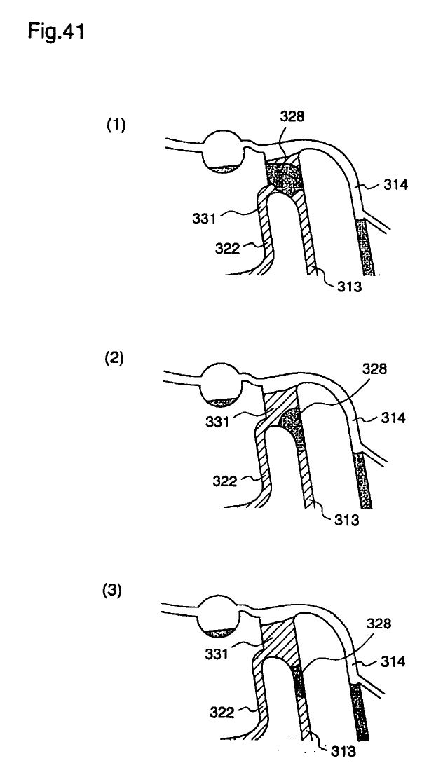

The state where the DNA sample migrates while being condensed is shown in (1)˜(3) in FIG. 41.

FIG. 41 shows the electrophoresis start initial state, wherein (1), (2), (3) show the states of the DNA sample 328 at 10 seconds, 20 seconds, and 30 seconds after voltage application, respectively. It can be seen from these figures that the DNA sample 328 gradually migrates to the channel 313. Since only the DNA sample migrates from a portion of a certain volume to the narrower channel, a phenomenon that the DNA sample is condensed occurs. That is, even a DNA sample or a low concentration that is hard to detect is condensed and easily detected when it reaches the channel 313.

Since the DNA conjugate is filled in the channel 313, the condensed DNA sample performs electrophoresis while repeating bonding with the DNA conjugate. At this time, since the bonding force of the normal DNA in the DNA sample with the DNA conjugate is strong as described above, the electrophoresis speed of the normal DNA is lowered. On the other hand, since the bonding force of the mutant DNA with the DNA conjugate is weak, the electrophoresis speed of the mutant DNA is higher than that of the normal DNA. That is, when both the normal DNA and the mutant DNA exist in the DNA sample, the normal DNA and the mutant DNA are separated, whereby discrimination of SNPs can be carried out.

DNA detection is carried out by exciting the fluorescent-labeled (FITC) DNA with light of 470 nm, and performing photo detection in the vicinity of 520 nm. This DNA detection may be carried out by detecting absorbance at 260 nm.

As described above, according to the fifth embodiment, the condensation part 330 in which the cross-sectional area of the channel decreases from the sample quantitation part 315 to the electrophoresis channel 313 for the buffer agent is provided on the biological sample analysis plate 500, and a predetermined amount of the DNA sample is stored in the sample quantitation part 315 by only the rotating operation of the plate 500, and the stored predetermined amount of DNA sample is made to perform electrophoresis by the positive electrode part 310, the negative electrode part 309, and the channel 313, and further, the channel 313 for the electrophoresis is shaped like a circular ark. Therefore, when measuring a DNA sample that is obtained by extracting specific DNA from cell or blood by using the biological sample analysis plate, even if the concentration of the DNA sample is low, extremely sensitive detection result can be obtained.

### Embodiment 6

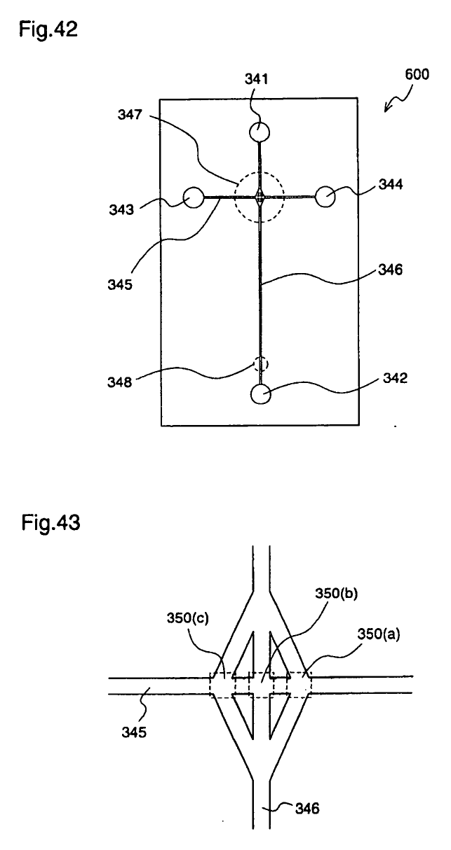

Hereinafter, a biological sample analysis plate according to a sixth embodiment will be described with reference to FIGS. 42˜45.

According to the present invention, a biological sample is migrated in a buffer agent to carry out biological, enzymatical, immunological, and chemical reactions, thereby to analyze the biological sample easily, inexpensively, accurately, and speedily.

Also in this sixth embodiment, presence/absence of SNPs (Single Nucleotide Polymorphism) in a DNA sample is determined using the same principle as that described for the fifth embodiment.

FIG. 42 is a diagram illustrating the biological sample analysis plate viewed from a channel formation surface thereof, according to the sixth embodiment. Hereinafter, the construction of the biological sample analysis plate 600 according to the sixth embodiment will be described.

The outer shape of the biological sample analysis plate 340 according to the sixth embodiment is 30 mm ×50 mm in size, and it is 2 mm thick. A 50 μm thick transparent film 50 is adhered to a surface of the plate where a channel 345 through which the biological sample flows and a channel 346 through which the buffer agent flows are formed.

All of buffer parts 341˜344 are through holes, and only one side of each through hole is hermetically sealed by the film.

The buffer part 343 and the buffer part 344 are connected by the channel 345, and the buffer part 341 and the buffer part 342 are connected by the channel 346 Further, the channel 345 and the channel 346 intersect with each other at a midpoint.

In this sixth embodiment, the channels are 50 μm deep, and 100-300 μm wide.

In the biological sample analysis plate 600 according to the sixth embodiment, a sample quantitation part 347 is disposed at a point where the channel 345 intersects with the channel 346, and an enlarged view of the sample quantitation part 347 and its vicinity is shown in FIG. 43.

FIG. 43 is an enlarge view of the sample quantitation part 347 and its vicinity, of the biological sample analysis plate 600 according to the sixth embodiment.

In FIG. 43, reference numerals 350(a), 350(b), and 350(c) are intersection points of the channel 345 with the channel 346, aid there are three intersection points because the channel 346 branches into three channels at a point near the intersection with the channel 345.

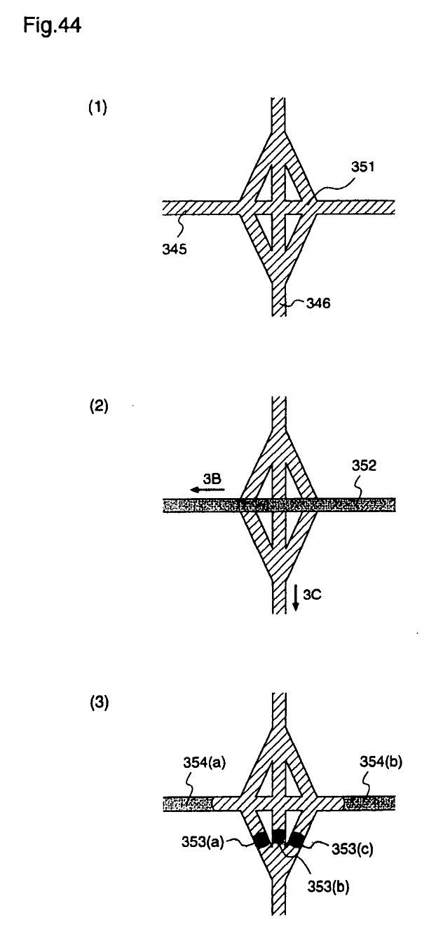

Next, a description will be given of the procedure and operation of experiment for separating SNPs, in the biological sample analysis plate 600 according to the sixth embodiment, with reference to FIGS. 44(1)˜(3) and 45.

FIGS. 44(1)˜(3) and 45 are enlarged views of the sample quantitation part and its vicinity, illustrating the DNA sample migration states after starting electrophoresis, in the biological sample analysis plate 600 according to the sixth embodiment.

As in the fifth embodiment mentioned above, after preparing the DNA sample and the DNA conjugate, the DNA conjugate is filled in the buffer part 342 and the channels 345 and 346. As a filling method, pressurization may be employed. Next, the DNA sample is injected into the buffer part 344, and the DNA conjugate is injected into the buffer part 343 and the buffer part 341. Thus, filling of the samples is completed.

FIG. 44(1) shows the state of the sample quantitation part 347 and its vicinity at this time.

In FIG. 44(1), the DNA conjugate 351 is filled in the entire channel.

Next, positive voltage is applied to the buffer part 343 with the buffer part 344 as a reference. Since the DNA is negatively charged, the DNA performs electrophoresis in the channel 345 toward the buffer part 343 (3B direction) to which the positive voltage is applied.

FIG. 44(2) shows the state of the sample quantitation part 347 and its vicinity after 400V is applied for three minutes.

The above-mentioned voltage application is once stopped, and negative voltage is again applied to the buffer parts 341, 343, axed 344 with the buffer part 342 as a reference. Then, as shown in FIG. 44(3), the DNA samples 353(a), 353(b), and 353(c) in the vicinity of the intersection point of the channel 345 with the channel 346 perform electrophoresis in the channel 346 toward the buffer part 342 (3C direction).

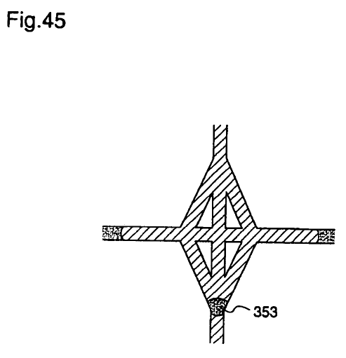

When the electrophoresis further proceeds, there is a portion where the three channels 346 converge to one channel, wherein the DNA samples 353(a), 353(b), and 353(c) intersect. FIG. 45 shows the state at this time, and the concentration of the DNA sample 353 in FIG. 45 is much higher than each of the concentrations of the DNA samples 353(a), 353(b), and 353(c) shown in FIG. 44(3). In FIG. 44(3), reference numerals 354(a) and 354(b) show the left-side portion and the right-side portion of the channel 354, respectively.

When the electrophoresis further proceeds, the DNA sample reacts with the buffer agent in the channel 346, whereby the normal DNA and the mutant DNA are separated. The DNA samples thus separated are detected when they reach the detection part 398.

As described above, according to the biological sample analysis plate 600 according to the sixth embodiment, the sample quantitation part is divided into plural parts, and the DNA samples in the respective parts are joined together. Therefore, the migrating DNA sample is condensed during electrophoresis, whereby highly sensitive detection can be carried out.

### APPLICABILITY IN INDUSTRY

The biological sample analysis plate and the biological sample analysis method according to the present invention are useful as those capable of performing analysis of a biological sample such as a DNA sample inexpensively and easily.

1. A biological sample analysis plate having a channel in which an injected buffer agent is filled by a centrifugal force that is generated by rotating the plate about its axial center, said channel comprising a first channel and a second channel, one ends of the said first and second channels being connected to a quantitative sorting part having a predetermined volume for taking a predetermined amount of a biological sample, and said first and second channels having a shape extending form the quantitative sorting part toward an outer circumference with respect to the axial center, said plate including:
a buffer agent storage part for holding the buffer agent that is filled in the channel, said buffer agent storage part being located at the other end of the first channel, in a position on a concentric circle with the quantitative sorting part with respect to the axial center, or in a position closer to the axial center than the quantitative sorting part.

2. A biological sample analysis plate as defined in claim 1 wherein
said second channel is connected to an overflow chamber into which excess buffer agent that cannot be stored in the buffer agent storage part flows.

3. A biological sample analysis plate as defined in claim 1 wherein
an upper-level buffer agent injection part for injecting the buffer agent is provided in a position closer to the axial center than the buffer agent storage part, and
said upper-level buffer agent injection part and said buffer agent storage part are connected to each other.

4. A biological sample analysis plate as defined in claim 3 wherein
a through-hole in which air comes in and out is provided in said channel or in said upper-level buffer agent injection part, thereby to promote filling of the buffer agent into the channel.

5. A biological sample analysis plate as defined in claim 1 wherein
a biological sample injection part for injecting the biological sample is connected to the quantitative sorting part; and
a through-hole in which air comes in and out is provided in the quantitative sorting parts so as to promote supple of the biological sample held by the biological sample injection part into the quantitative sorting part.

6. A biological sample analysis plate as defined in any of claim 1 wherein
said quantitative sorting part is connected to an overflow chamber into which the biological sample that is supplied over a predetermined amount flow.

7. A biological sample analysis plate in which a quantitative sorting part for introducing a predetermined amount of a biological sample into a buffer agent channel is provided at a junction of a sample channel in which the biological sample flows and the buffer agent channel in which a buffer agent flows, said plate performing analysis of the biological sample by detecting a transport reaction that occurs when the biological sample introduced in the quantitative sorting part migrated in the buffer agent wherein,
said sample channel is provided on an inner circumference side of a closed channel which forms the buffer agent channel.

8. A biological sample analysis plate as defined in claim 7 wherein
the length of a portion of the buffer agent channel, which portion is formed from the center of the biological sample analysis plate toward the outer circumference, is longer than the length of a portion of the buffer agent channel, which portion is formed in a rotation direction of the plate around the center of the plate.

9. A biological sample analysis plate as defined in claim 8 wherein
said buffer agent channel has a rectangle shape.

10. A biological sample analysis plate as defined in claim 8 wherein
said buffer agent channel is disposed on a centric circle with the gravity center of the biological sample analysis plate as a center of the rotation.

11. A biological sample analysis plate as defined in claim 10 wherein
a cross-sectional area of a portion of the buffer agent channel on the inner circumference side is larger than the cross-sectional area of a portion of the buffer agent channel on the outer circumference side.

12. A biological sample analysis plate for detecting a transport reaction that occurs when a biological sample migrates in a buffer agent, thereby to analyze the biological sample, said plate including:
a quantitative sorting part having a predetermined volume for holding the biological sample, which is provided at a junction of a third channel in which the buffer agent flows and a fourth channel in which the biological sample flows; and
a suppression means for suppressing flow of the biological sample held by the quantitative sorting part into the fourth channel, which is provided in the quantitative sorting part.

13. A biological sample analysis plate as defined in claim 12 wherein
a cross-sectional area of a portion of the fourth channel for making the biological sample flow into the quantitative sorting part is equal to or larger than a cross-sectional area of a portion of the fourth channel for making the biological sample flow from the quantitative sorting part.

14. A biological sample analysis plate as defined in claim 12 wherein
a cross-sectional area of a portion of the fourth channel which is a disposed above the suppression means in the quantitative sorting part is equal to or smaller than a cross-sectional area of a portion between the suppressing means and a side wall surface of the quantitative sorting part, which portion makes the biological sample flow from the fourth channel into the quantitative sorting part, and is equal to or smaller than a cross-sectional area of a portion between the suppressing means and the side wall surface of the quantitative sorting part, which portion makes the biological sample flow from the quantitative sorting part to the fourth channel.

15. A biological sample analysis plate as defined in claim 12 wherein
said quantitative sorting part is provided with a guide means for guiding the biological sample into the third channel.

16. A biological sample analysis plate as defined in claim 15 wherein
said guide means is protruding portion of the quantitative sorting.

17. A biological sample analysis plate as defined in claim 12 wherein
the cross-sectional area of the third channel decreased in proportion to the distance from the quantitative sorting part.

18. A biological sample analysis plate for detecting a transport reaction that occurs when a biological sample migrates in a buffer agent, thereby to perform analysis of the biological sample, said plate including:
a fifth channel in which the biological sample flows, a sixth channel in which the buffer agent flows, and a quantitative sorting part for holding a predetermined volume of the biological sample, which part is disposed at a junction of the fifth channel and the sixth channel;
wherein, among channels at the junction of the quantitative sorting part and the sixth channel, a cross-sectional area of a sample flow-in channel through which the biological sample flows from the quantitative sorting part of the sixth channel decreases in proportion to the distance from the quantitative sorting part.

19. A biological sample analysis plate as defined in claim 18 wherein
said quantitative sorting part sorting comprises plural parts, and the biological sample flows from the plural parts into the flow-in channel.

20. A biological sample analysis plate as defined in claim 19 wherein
said sixth channel comprises plural channels.
